(19) United States
(12) Patent Application Publication
(10) Pub. No.: US 2024/0318265 A1
(43) Pub. Date: Sep. 26, 2024

(54) LACTIC ACID BACTERIUM DETECTION PRIMER SET, AND DETECTION METHOD USING SAID PRIMER SET

(71) Applicant: NIHON BERUMU CO., LTD., Tokyo (JP)

(72) Inventors: Akihiko OGASAWARA, Tokyo (JP); Masahiro IWASA, Tokyo (JP)

(73) Assignee: NIHON BERUMU CO., LTD., Tokyo (JP)

(21) Appl. No.: 18/259,433

(22) PCT Filed: Feb. 10, 2022
(86) PCT No.: PCT/JP2022/005426

§ 371 Date: Jun. 27, 2023

(30) Foreign Application Priority Data
Feb. 12, 2021 (JP) 2021-021102

(51) Int. Cl. C12Q 1/689; C12N 1/20; C12Q 1/6844; C12R 1/46

(52) U.S. Cl. CPC C12Q 1/689; C12N 1/205; C12Q 1/6844; C12R 2001/46

(57) ABSTRACT

The present invention provides: a primer set capable of identifying lactic acid bacterium EF-2001 strain specifically; and a detection method using the primer set.

According to the present invention, a primer set for use in the detection of lactic acid bacterium EF-2001 strain is provided, which comprises oligonucleotides comprising a combination of SEQ ID NOs: 1 and 2, SEQ ID NOs: 4 and 5, SEQ ID NOs: 7 and 8, SEQ ID NOs: 10 and 11, SEQ ID NOs: 13 and 14, SEQ ID NOs: 16 and 17, SEQ ID NOs: 19 and 20, SEQ ID NOs: 22 and 23, SEQ ID NOs: 25 and 26, and/or SEQ ID NOs: 28 and 29, or oligonucleotides comprising a combination of SEQ ID NOs: 1 and 3, SEQ ID NOs: 4 and 6, SEQ ID NOs: 7 and 9, SEQ ID NOs: 10 and 12, SEQ ID NOs: 13 and 15, SEQ ID NOs: 16 and 18, SEQ ID NOs: 19 and 21, SEQ ID NOs: 22 and 24, SEQ ID NOs: 25 and 27, and/or SEQ ID NOs: 28 and 30.

### TECHNICAL FIELD

The present invention relates to a method for detecting a specific lactic acid bacterium. Specifically, the present invention relates to a primer set for detecting an Enterococcus faecalis EF-2001 strain and a detection method using the primer set.

### BACKGROUND ART

A lactic acid bacterium is known to have various actions. In recent years, a lactic acid bacterium is utilized as a useful microorganism resource deeply involved in humans' health because a lactic acid bacterium has various health retaining effects such as the effect of regulating the intestinal function by improving a bacterial flora in the gastrointestinal tract as probiotics, the immunity improving effect, or the anti-tumor effect.

Meanwhile, Enterococcus faecalis, which is one type of lactic acid bacterium, also includes a bacterial cell that is known to cause an opportunistic infection. Such a bacterial cell may cause, as an opportunistic pathogen, various diseases including a urinary tract infectious disease and a life-threatening infectious disease such as bacteremia, endocarditis, or meningitis. Thus, it is extremely important to distinguish a bacterial cell which can be utilized as probiotics from a pathogenic bacterial cell.

### SUMMARY OF INVENTION

### Solution to Problem

It is reported that an EF-2001 strain, which is one Enterococcus faecalis strain, has various biological modulating activities including anti-inflammation and immunomodulation. Said strain is widely utilized as probiotics of supplements or functional food. The present invention provides a primer set which can rapidly and simply perform specific identification of a lactic acid bacterium Enterococcus faecalis EF-2001 strain, and a detection method using the primer set.

Thus, the present disclosure provides the following items.

### (Item 1)

A primer set for specifically detecting a nucleic acid derived from a lactic acid bacterium Enterococcus faecalis EF-2001 strain, the primer set comprising:
a pair of an oligonucleotide comprising the base sequence of SEQ ID NO: 1 and an oligonucleotide comprising the base sequence of SEQ ID NO: 2 or 3;
a pair of an oligonucleotide comprising the base sequence of SEQ ID NO: 4 and an oligonucleotide comprising the base sequence of SEQ ID NO: 5 or 6;
a pair of an oligonucleotide comprising the base sequence of SEQ ID NO: 7 and an oligonucleotide comprising the base sequence of SEQ ID NO: 8 or 9;
a pair of an oligonucleotide comprising the base sequence of SEQ ID NO: 10 and an oligonucleotide comprising the base sequence of SEQ ID NO: 11 or 12;
a pair of an oligonucleotide comprising the base sequence of SEQ ID NO: 13 and an oligonucleotide comprising the base sequence of SEQ ID NO: 14 or 15;
a pair of an oligonucleotide comprising the base sequence of SEQ ID NO: 16 and an oligonucleotide comprising the base sequence of SEQ ID NO: 17 or 18;
a pair of an oligonucleotide comprising the base sequence of SEQ ID NO: 19 and an oligonucleotide comprising the base sequence of SEQ ID NO: 20 or 21;
a pair of an oligonucleotide comprising the base sequence of SEQ ID NO: 22 and an oligonucleotide comprising the base sequence of SEQ ID NO: 23 or 24;
a pair of an oligonucleotide comprising the base sequence of SEQ ID NO: 25 and an oligonucleotide comprising the base sequence of SEQ ID NO: 26 or 27; or
a pair of an oligonucleotide comprising the base sequence of SEQ ID NO: 28 and an oligonucleotide comprising the base sequence of SEQ ID NO: 29 or 30.

### (Item 2)

A primer set for specifically detecting a nucleic acid derived from a lactic acid bacterium Enterococcus faecalis EF-2001 strain, the primer set selected from the group consisting of
(a) a primer set (1) for amplifying a part of the base sequence of SEQ ID NO: 51:

a forward primer
(SEQ ID NO: 1)
CGAAAAGGATGTAGTCAGCGG;
and
a reverse primer
(SEQ ID NO: 2)
CCGAAGGCGAAACAGAGGAT,

(b) a primer set (2) for amplifying a part of the base sequence of SEQ ID NO: 51:

a forward primer
(SEQ ID NO: 1)
CGAAAAGGATGTAGTCAGCGG;
and
a reverse primer
(SEQ ID NO: 3)
CCCAGACATAATCGCATGGC,

(c) a primer set (3) for amplifying a part of the base sequence of SEQ ID NO: 52:

a forward primer
(SEQ ID NO: 4)
GCGGCTGCACAATTTATTGC;
and
a reverse primer
(SEQ ID NO: 5)
AGAATACTTGGGCGGTCGTG,

(d) a primer set (4) for amplifying a part of the base sequence of SEQ ID NO: 52:

a forward primer
(SEQ ID NO: 4)
GCGGCTGCACAATTTATTGC;
and
a reverse primer
(SEQ ID NO: 6)
AATTCAGCTTCGCTAGATAAGGC,

(e) a primer set (5) for amplifying a part of the base sequence of SEQ ID NO: 53:

a forward primer
(SEQ ID NO: 7)
CGCGTATGACTTGCAATCGA;
and
a reverse primer
(SEQ ID NO: 8)
AGGATTGTTTGACGGTGCAA,

(f) a primer set (6) for amplifying a part of the base sequence of SEQ ID NO: 53:

a forward primer
(SEQ ID NO: 7)
CGCGTATGACTTGCAATCGA,
and
a reverse primer
(SEQ ID NO: 9)
ACATGAGATAGTTGGGGTAGACA;

(g) a primer set (7) for amplifying a part of the base sequence of SEQ ID NO: 54:

a forward primer
(SEQ ID NO: 10)
CTTCAGAGAGCTGGGCGAAG;
and
a reverse primer
(SEQ ID NO: 11)
TACTTTTTAGCTGCCCGCCC,

(h) a primer set (8) for amplifying a part of the base sequence of SEQ ID NO: 54:

a forward primer
(SEQ ID NO: 10)
CTTCAGAGAGCTGGGCGAAG;
and
a reverse primer
(SEQ ID NO: 12)
GGGTTGTAGCCCTACCCGAT,

(i) a primer set (9) for amplifying a part of the base sequence of SEQ ID NO: 55:

a forward primer
(SEQ ID NO: 13)
CGTAACGTGACATTGCGGAC;
and
a reverse primer
(SEQ ID NO: 14)
ATGCCAGTACGTCGCGTTAA,

(j) a primer set (10) for amplifying a part of the base sequence of SEQ ID NO: 55:

a forward primer
(SEQ ID NO: 13)
CGTAACGTGACATTGCGGAC;
and
a reverse primer
(SEQ ID NO: 15)
CGATTGTCAACTAATTGTGCCGA,

(k) a primer set (11) for amplifying a part of the base sequence of SEQ ID NO: 56:

a forward primer
(SEQ ID NO: 16)
CATGGCTTGCCGTTTCACAA;
and
a reverse primer
(SEQ ID NO: 17)
ACCGCAACAACTACATACTACCA,

(1) a primer set (12) for amplifying a part of the base sequence of SEQ ID NO: 56:

a forward primer
(SEQ ID NO: 16)
CATGGCTTGCCGTTTCACAA;
and
a reverse primer
(SEQ ID NO: 18)
ACCAAAAGGAACGCTACCAGT,

(m) a primer set (13) for amplifying a part of the base sequence of SEQ ID NO: 57:

a forward primer
(SEQ ID NO: 19)
TCAGCATAATCCCCAGACGT;
and
a reverse primer
(SEQ ID NO: 20)
AATGAACGCCCTTCAGCAGA,

(n) a primer set (14) for amplifying a part of the base sequence of SEQ ID NO: 57:

a forward primer
(SEQ ID NO: 19)
TCAGCATAATCCCCAGACGT;
and
a reverse primer
(SEQ ID NO: 21)
GGCTCCTCTACCTGAACAAACT,

(o) a primer set (15) for amplifying a part of the base sequence of SEQ ID NO: 58:

a forward primer
(SEQ ID NO: 22)
GCGTTCAAACTGTTCTGGTGT;
and
a reverse primer
(SEQ ID NO: 23)
TACAAGGCTTGCGAGGTAGC,

(p) a primer set (16) for amplifying a part of the base sequence of SEQ ID NO: 58:

a forward primer
(SEQ ID NO: 22)
GCGTTCAAACTGTTCTGGTGT;
and
a reverse primer
(SEQ ID NO: 24)
GCTGCAATGGAAAGCAAATCG,

(q) a primer set (17) for amplifying a part of the base sequence of SEQ ID NO: 59:

a forward primer
(SEQ ID NO: 25)
AGGCATATGGGTCATCTGCT;
and
a reverse primer
(SEQ ID NO: 26)
GAGCATCACAGAGCCTCGAA,

(r) a primer set (18) for amplifying a part of the base sequence of SEQ ID NO: 59:

a forward primer
(SEQ ID NO: 25)
AGGCATATGGGTCATCTGCT;
and
a reverse primer
(SEQ ID NO: 27)
AGAGATTTTTCAGTATTGCTGGGT,

(s) a primer set (19) for amplifying a part of the base sequence of SEQ ID NO: 60:
   a forward primer CGTTGGGTGTGCAGAAATGG (SEQ ID NO: 28); and
   a reverse primer TGTACCGTCAACCTCGTTCG (SEQ ID NO: 29), or
(t) a primer set (20) for amplifying a part of the base sequence of SEQ ID NO: 60:

a forward primer
(SEQ ID NO: 28)
CGTTGGGTGTGCAGAAATGG;
and
a reverse primer
(SEQ ID NO: 30)
AACGGGTTGCGACTCTTTTT.

### (Item 3)

A kit for detecting a lactic acid bacterium Enterococcus faecalis EF-2001 strain, characterized by comprising the primer set of item 1 or 2.

### (Item 4)

A method for specifically detecting a nucleic acid derived from a lactic acid bacterium Enterococcus faecalis EF-2001 strain, the method comprising:
the step of amplifying a nucleic acid fragment using the primer set of item 1 or 2; and
the step of detecting a nucleic acid fragment obtained in the amplification step.

### (Item 5)

A lactic acid bacterium composition comprising a nucleic acid fragment,
wherein the nucleic acid fragment is not amplified by one or more primer sets selected from the group consisting of:
a pair of an oligonucleotide comprising the base sequence of SEQ ID NO: 1 and an oligonucleotide comprising the base sequence of SEQ ID NO: 2;
a pair of an oligonucleotide comprising the base sequence of SEQ ID NO: 4 and an oligonucleotide comprising the base sequence of SEQ ID NO: 5;
a pair of an oligonucleotide comprising the base sequence of SEQ ID NO: 7 and an oligonucleotide comprising the base sequence of SEQ ID NO: 8;
a pair of an oligonucleotide comprising the base sequence of SEQ ID NO: 10 and an oligonucleotide comprising the base sequence of SEQ ID NO: 11;
a pair of an oligonucleotide comprising the base sequence of SEQ ID NO: 13 and an oligonucleotide comprising the base sequence of SEQ ID NO: 14;
a pair of an oligonucleotide comprising the base sequence of SEQ ID NO: 16 and an oligonucleotide comprising the base sequence of SEQ ID NO: 17;
a pair of an oligonucleotide comprising the base sequence of SEQ ID NO: 19 and an oligonucleotide comprising the base sequence of SEQ ID NO: 20;
a pair of an oligonucleotide comprising the base sequence of SEQ ID NO: 22 and an oligonucleotide comprising the base sequence of SEQ ID NO: 23;
a pair of an oligonucleotide comprising the base sequence of SEQ ID NO: 25 and an oligonucleotide comprising the base sequence of SEQ ID NO: 26; and/or
a pair of an oligonucleotide comprising the base sequence of SEQ ID NO: 28 and an oligonucleotide comprising the base sequence of SEQ ID NO: 29, and
wherein the nucleic acid fragment is amplified by one or more primer sets selected from the group consisting of:
a pair of an oligonucleotide comprising the base sequence of SEQ ID NO: 1 and an oligonucleotide comprising the base sequence of SEQ ID NO: 3;
a pair of an oligonucleotide comprising the base sequence of SEQ ID NO: 4 and an oligonucleotide comprising the base sequence of SEQ ID NO: 6;
a pair of an oligonucleotide comprising the base sequence of SEQ ID NO: 7 and an oligonucleotide comprising the base sequence of SEQ ID NO: 9;
a pair of an oligonucleotide comprising the base sequence of SEQ ID NO: 10 and an oligonucleotide comprising the base sequence of SEQ ID NO: 12;
a pair of an oligonucleotide comprising the base sequence of SEQ ID NO: 13 and an oligonucleotide comprising the base sequence of SEQ ID NO: 15;
a pair of an oligonucleotide comprising the base sequence of SEQ ID NO: 16 and an oligonucleotide comprising the base sequence of SEQ ID NO: 18;
a pair of an oligonucleotide comprising the base sequence of SEQ ID NO: 19 and an oligonucleotide comprising the base sequence of SEQ ID NO: 21;
a pair of an oligonucleotide comprising the base sequence of SEQ ID NO: 22 and an oligonucleotide comprising the base sequence of SEQ ID NO: 24;
a pair of an oligonucleotide comprising the base sequence of SEQ ID NO: 25 and an oligonucleotide comprising the base sequence of SEQ ID NO: 27; and/or
a pair of an oligonucleotide comprising the base sequence of SEQ ID NO: 28 and an oligonucleotide comprising the base sequence of SEQ ID NO: 30.

### (Item 6)

The composition of item 5, comprising a nucleic acid fragment which is not amplified by a primer set of an oligonucleotide comprising the base sequence of SEQ ID NO: 16 and an oligonucleotide comprising the base sequence of SEQ ID NO: 17 and is amplified by a primer set of an oligonucleotide comprising the base sequence of SEQ ID NO: 16 and an oligonucleotide comprising the base sequence of SEQ ID NO: 18.

### (Item 7)

The composition of item 5 or 6, comprising a heat-treated lactic acid bacterium product resulting from subjecting a lactic acid bacterium Enterococcus faecalis EF-2001 strain to heat treatment at at least about 70° C.

### (Item 8)

The composition of any one of items 5 to 7, comprising a heat-treated lactic acid bacterium product resulting from subjecting a lactic acid bacterium Enterococcus faecalis EF-2001 strain to heat treatment at at least about 90° C.

### (Item 9)

A method for manufacturing a lactic acid bacterium composition, the method comprising:
the step of heating a lactic acid bacterium Enterococcus faecalis EF-2001 strain to obtain a plurality of lots of a heat-treated lactic acid bacterium product;
the step of extracting a nucleic acid fragment in one of the lots of the heat-treated lactic acid bacterium product;
the step of amplifying the nucleic acid fragment using at least one of the primer sets of item 1 or 2;
the step of detecting a nucleic acid fragment obtained in the amplification step; and
the step of selecting a lot of the heat-treated lactic acid bacterium product in which the nucleic acid fragment has been detected.

### (Item 10)

The method of item 9, wherein the heating is heating at at least about 70° C.

### (Item 11)

The method of item 9 or 10, wherein the heating is heating at at least about 90° C.

### (Item 12)

The method of any one of items 9 to 11, wherein the nucleic acid fragment is amplified by one or more primer sets selected from the group consisting of:
a pair of an oligonucleotide comprising the base sequence of SEQ ID NO: 1 and an oligonucleotide comprising the base sequence of SEQ ID NO: 3;
a pair of an oligonucleotide comprising the base sequence of SEQ ID NO: 4 and an oligonucleotide comprising the base sequence of SEQ ID NO: 6;
a pair of an oligonucleotide comprising the base sequence of SEQ ID NO: 7 and an oligonucleotide comprising the base sequence of SEQ ID NO: 9;
a pair of an oligonucleotide comprising the base sequence of SEQ ID NO: 10 and an oligonucleotide comprising the base sequence of SEQ ID NO: 12;
a pair of an oligonucleotide comprising the base sequence of SEQ ID NO: 13 and an oligonucleotide comprising the base sequence of SEQ ID NO: 15;
a pair of an oligonucleotide comprising the base sequence of SEQ ID NO: 16 and an oligonucleotide comprising the base sequence of SEQ ID NO: 18;
a pair of an oligonucleotide comprising the base sequence of SEQ ID NO: 19 and an oligonucleotide comprising the base sequence of SEQ ID NO: 21;
a pair of an oligonucleotide comprising the base sequence of SEQ ID NO: 22 and an oligonucleotide comprising the base sequence of SEQ ID NO: 24;
a pair of an oligonucleotide comprising the base sequence of SEQ ID NO: 25 and an oligonucleotide comprising the base sequence of SEQ ID NO: 27; and/or
a pair of an oligonucleotide comprising the base sequence of SEQ ID NO: 28 and an oligonucleotide comprising the base sequence of SEQ ID NO: 30, and
wherein the nucleic acid fragment is not amplified by one or more primer sets selected from the group consisting of:
a pair of an oligonucleotide comprising the base sequence of SEQ ID NO: 1 and an oligonucleotide comprising the base sequence of SEQ ID NO: 2;
a pair of an oligonucleotide comprising the base sequence of SEQ ID NO: 4 and an oligonucleotide comprising the base sequence of SEQ ID NO: 5;
a pair of an oligonucleotide comprising the base sequence of SEQ ID NO: 7 and an oligonucleotide comprising the base sequence of SEQ ID NO: 8;
a pair of an oligonucleotide comprising the base sequence of SEQ ID NO: 10 and an oligonucleotide comprising the base sequence of SEQ ID NO: 11;
a pair of an oligonucleotide comprising the base sequence of SEQ ID NO: 13 and an oligonucleotide comprising the base sequence of SEQ ID NO: 14;
a pair of an oligonucleotide comprising the base sequence of SEQ ID NO: 16 and an oligonucleotide comprising the base sequence of SEQ ID NO: 17;
a pair of an oligonucleotide comprising the base sequence of SEQ ID NO: 19 and an oligonucleotide comprising the base sequence of SEQ ID NO: 20;
a pair of an oligonucleotide comprising the base sequence of SEQ ID NO: 22 and an oligonucleotide comprising the base sequence of SEQ ID NO: 23;
a pair of an oligonucleotide comprising the base sequence of SEQ ID NO: 25 and an oligonucleotide comprising the base sequence of SEQ ID NO: 26; and/or
a pair of an oligonucleotide comprising the base sequence of SEQ ID NO: 28 and an oligonucleotide comprising the base sequence of SEQ ID NO: 29.

### (Item 13)

A polynucleotide characterized by being amplified by a primer set for detecting a lactic acid bacterium Enterococcus faecalis EF-2001 strain, wherein the polynucleotide is:
the polynucleotide set forth in SEQ ID NO: 31 which is amplified by a primer set consisting of a pair of an oligonucleotide comprising the base sequence of SEQ ID NO: 1 and an oligonucleotide comprising the base sequence of SEQ ID NO: 2;
the polynucleotide set forth in SEQ ID NO: 32 which is amplified by a primer set consisting of a pair of an oligonucleotide comprising the base sequence of SEQ ID NO: 1 and an oligonucleotide comprising the base sequence of SEQ ID NO: 3;
the polynucleotide set forth in SEQ ID NO: 33 which is amplified by a primer set consisting of a pair of an oligonucleotide comprising the base sequence of SEQ ID NO: 4 and an oligonucleotide comprising the base sequence of SEQ ID NO: 5;
the polynucleotide set forth in SEQ ID NO: 34 which is amplified by a primer set consisting of a pair of an oligonucleotide comprising the base sequence of SEQ ID NO: 4 and an oligonucleotide comprising the base sequence of SEQ ID NO: 6;
the polynucleotide set forth in SEQ ID NO: 35 which is amplified by a primer set consisting of a pair of an oligonucleotide comprising the base sequence of SEQ ID NO: 7 and an oligonucleotide comprising the base sequence of SEQ ID NO: 8;
the polynucleotide set forth in SEQ ID NO: 36 which is amplified by a primer set consisting of a pair of an oligonucleotide comprising the base sequence of SEQ ID NO: 7 and an oligonucleotide comprising the base sequence of SEQ ID NO: 9;
the polynucleotide set forth in SEQ ID NO: 37 which is amplified by a primer set consisting of a pair of an oligonucleotide comprising the base sequence of SEQ ID NO: 10 and an oligonucleotide comprising the base sequence of SEQ ID NO: 11;
the polynucleotide set forth in SEQ ID NO: 38 which is amplified by a primer set consisting of a pair of an oligonucleotide comprising the base sequence of SEQ ID NO: 10 and an oligonucleotide comprising the base sequence of SEQ ID NO: 12;
the polynucleotide set forth in SEQ ID NO: 39 which is amplified by a primer set consisting of a pair of an oligonucleotide comprising the base sequence of SEQ ID NO: 13 and an oligonucleotide comprising the base sequence of SEQ ID NO: 14;
the polynucleotide set forth in SEQ ID NO: 40 which is amplified by a primer set consisting of a pair of an oligonucleotide comprising the base sequence of SEQ ID NO: 13 and an oligonucleotide comprising the base sequence of SEQ ID NO: 15;
the polynucleotide set forth in SEQ ID NO: 41 which is amplified by a primer set consisting of a pair of an oligonucleotide comprising the base sequence of SEQ ID NO: 16 and an oligonucleotide comprising the base sequence of SEQ ID NO: 17;
the polynucleotide set forth in SEQ ID NO: 42 which is amplified by a primer set consisting of a pair of an oligonucleotide comprising the base sequence of SEQ ID NO: 16 and an oligonucleotide comprising the base sequence of SEQ ID NO: 18;
the polynucleotide set forth in SEQ ID NO: 43 which is amplified by a primer set consisting of a pair of an oligonucleotide comprising the base sequence of SEQ ID NO: 19 and an oligonucleotide comprising the base sequence of SEQ ID NO: 20;
the polynucleotide set forth in SEQ ID NO: 44 which is amplified by a primer set consisting of a pair of an oligonucleotide comprising the base sequence of SEQ ID NO: 19 and an oligonucleotide comprising the base sequence of SEQ ID NO: 21;
the polynucleotide set forth in SEQ ID NO: 45 which is amplified by a primer set consisting of a pair of an oligonucleotide comprising the base sequence of SEQ ID NO: 22 and an oligonucleotide comprising the base sequence of SEQ ID NO: 23;
the polynucleotide set forth in SEQ ID NO: 46 which is amplified by a primer set consisting of a pair of an oligonucleotide comprising the base sequence of SEQ ID NO: 22 and an oligonucleotide comprising the base sequence of SEQ ID NO: 24;
the polynucleotide set forth in SEQ ID NO: 47 which is amplified by a primer set consisting of a pair of an oligonucleotide comprising the base sequence of SEQ ID NO: 25 and an oligonucleotide comprising the base sequence of SEQ ID NO: 26;
the polynucleotide set forth in SEQ ID NO: 48 which is amplified by a primer set consisting of a pair of an oligonucleotide comprising the base sequence of SEQ ID NO: 25 and an oligonucleotide comprising the base sequence of SEQ ID NO: 27;
the polynucleotide set forth in SEQ ID NO: 49 which is amplified by a primer set consisting of a pair of an oligonucleotide comprising the base sequence of SEQ ID NO: 28 and an oligonucleotide comprising the base sequence of SEQ ID NO: 29; or
the polynucleotide set forth in SEQ ID NO: 50 which is amplified by a primer set consisting of a pair of an oligonucleotide comprising the base sequence of SEQ ID NO: 28 and an oligonucleotide comprising the base sequence of SEQ ID NO: 30.

The present disclosure is intended so that one or more of the aforementioned features can be provided not only as the explicitly shown combinations, but also as other combinations thereof. Additional embodiments and advantages of the present disclosure are recognized by those skilled in the art by reading and understanding the following detailed description, as needed.

Features other than those described above and significant action/effect of the present disclosure become clear to those skilled in the art by referring to the following section of the embodiments and the drawings.

### Advantageous Effects of Invention

The present invention can simply and rapidly detect a lactic acid bacterium Enterococcus faecalis EF-2001 strain without recognizing a bacterial strain closely related to a lactic acid bacterium Enterococcus faecalis EF-2001 strain. This is very useful.

### BRIEF DESCRIPTION OF DRAWINGS

FIG. 1 shows the results of electrophoresis showing the results of detecting a nucleic acid using the primer sets of one embodiment of the present invention and using the DNA of a lactic acid bacterium EF-2001 strain and various Enterococcus faecalis subspecies as a template. (A) A lactic acid bacterium EF-2001 strain, and (B) E. faecalis NBRC3971, (C) E. faecalis NBRC3989, (D) E. faecalis NBRC12970, (E) E. faecalis NBRC100480, (F) E. faecalis NBRC100482, (G) E. faecalis NBRC100483 and (H) E. faecalis NBRC100484, which are Enterococcus faecalis subspecies.

FIG. 2 shows the results of electrophoresis showing the results of detecting a nucleic acid using the primer sets of one embodiment of the present invention and using the DNA of a lactic acid bacterium EF-2001 strain and various other lactic acid bacteria as a template. (B) E. faecium Aus0004, (C) E. gallinarum LMG13129, (D) Lactobacillus rhamnosus NBRC3425, (E) L. plantarum NBRC15891, (F) L. paracasei NBRC15906, and (G) L. lactis NBRC102622.

FIG. 3 shows the results of electrophoresis showing the results of detecting a live bacterium and a dead bacterium using the primer sets (long and short) of one embodiment of the present invention and using the DNA of manufacturing step liquid of a lactic acid bacterium EF-2001 strain as a template. (A) Culture liquid (live bacterium), (B) liquid after heat treatment (dead bacterium), and (C) product bulk (dead bacterium).

FIG. 4 shows the results of electrophoresis confirming the disappearance of a band of the primer set of the present invention due to heat treatment by using a lactic acid bacterium EF-2001 strain.

FIG. 5 shows a graph confirming the change in the TNF-α production amount of a lactic acid bacterium EF-2001 strain due to heat treatment.

### DESCRIPTION OF EMBODIMENTS

The present disclosure is described hereinafter while showing the best mode thereof. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

The definitions of the terms and/or basic technical concepts that are particularly used herein are described hereinafter when appropriate.

As used herein, “about” means±10% of a numerical value that follows.

### PREFERRED EMBODIMENTS

Preferred embodiments of the present disclosure are described below. Embodiments provided below are provided to facilitate the understanding of the present disclosure. The scope of the present disclosure should not be limited to the following descriptions. Thus, it is apparent that those skilled in the art can make appropriate modifications within the scope of the present disclosure by referring to the descriptions herein. The following embodiments of the present disclosure can be used alone or in combination.

The primer set of the present invention can amplify a chromosomal DNA region having a base sequence specific to a lactic acid bacterium Enterococcus faecalis EF-2001 strain by PCR (polymerase chain reaction), and can specifically recognize and detect a lactic acid bacterium Enterococcus faecalis EF-2001 strain without recognizing a bacterial strain closely related to a lactic acid bacterium Enterococcus faecalis EF-2001 strain.

The primer set of the present invention was designed by the following method.

First, the sequence of a full length genome of a lactic acid bacterium EF-2001 strain was determined by using a hybrid assembly approach using Short Read from ThermoFisher IonPGM and Long Read from OxfordNanopore MinION. When the Draft Genome sequence obtained from this Short Read was determined, gene fragments of 1 kbp or greater which did not match when compared with 42 strains of Table 1 below were narrowed as a gene sequence specific to an EF-2001 strain.

TABLE 1
Enterococcus faecalis Complete Genome sequence information
of 42 strains used for comparison GenBank (dated Sep. 1, 2020)

| E. faecalis strain | Host/Source | Accession | Collection date |
|---|---|---|---|
| EF-2001 | Human | [symbol] | 1995 |
| R712 | Human | GCA_0096624 [symbol] .1 | missing |
| C25 | Pig | GCA_00597 [symbol] 285.1 | 2017 |
| [symbol] 1RF | missing | GCA_000172575.2 | missing |
| [symbol] | missing | GCA_000211255.2 | missing |
| DEN [symbol] | Human | GCA_000 [symbol] .1 | missing |
| ATCC29212 | missing | GCA_00 [symbol] .1 | missing |
| 111540027-2 | Environment (ISS) | GCA_013344645.1 | 2011 |
| 111540027-1 | Environment (ISS) | GCA_013344625.1 | 2011 |
| NCT [symbol] | missing | GCA_ [symbol] | missing |
| FDAA [symbol] | Human | GCA_00220 [symbol] | 20 [symbol] |
| NCT [symbol] | missing | GCA_901563725.1 | missing |
| FDAA [symbol] | Human | GCA_003030425.1 | 2015 |
| W11 | Food | GCA_00 [symbol] .1 | missing |
| A [symbol] | missing | GCA_00 [symbol] 115.1 | 2001 |
| SGA [symbol] | Environment (air) | GCA_00548 [symbol] | 2015 |
| FDAA [symbol] | Food | GCA_00 [symbol] | missing |
| D32 | Pig | GCA_000 [symbol] 95.1 | 2001 |
| 133170041-3 | Environment (air at ISS) | GCA_0133 [symbol] .1 | 2013 |
| C [symbol] | Pig | GCA_00 [symbol] | 2017 |
| Symbio [symbol] | missing | GCA_00 [symbol] | missing |
| [symbol] | Food | GCA_00 [symbol] | 2010 |
| KUB [symbol] | Human | GCA_00 [symbol] | 2017 |
| KUB [symbol] | Human | GCA_00 [symbol] | 2017 |
| FC | Milk | GCA_00 [symbol] | 2017 |
| H2 [symbol] | missing | GCA_00228 [symbol] .2 | missing |
| K [symbol] 1 | Mouse | GCA_00 [symbol] | 201 [symbol] |
| L8 | Pig | GCA_00 [symbol] | 2013 |
| [symbol] | Human | GCA_012594 [symbol] .1 | missing |
| [symbol] A-1 | Human | GCA_00 [symbol] .1 | 2019 |
| JY32 | Human | GCA_01010 [symbol] .1 | 201 [symbol] |
| L15 | Pig | GCA_0094 [symbol] | 201 [symbol] |
| L12 | Pig | GCA_00188 [symbol] 75.1 | 201 [symbol] |
| E [symbol] | Pig | GCA_011193375.1 | 2017 |
| V [symbol] | missing | GCA_0000077 [symbol] .1 | missing |
| [symbol] | Fish | GCA_00 [symbol] | 2018 |
| [symbol] | Human | GCA_00 [symbol] 735.1 | 2014 |
| [symbol] | Environment (air at ISS) | GCA_0133 [symbol] .1 | 2009 |
| D [symbol] | Food | GCA_00 [symbol] 146.1 | 201 [symbol] |
| FDAA [symbol] | Human | GCA_00 [symbol] 665.1 | 2016 |
| CLB21560 | Human | GCA_00 [symbol] 555.1 | 2017 |
| [symbol] | Fish | GCA_003345275.1 | 2011 |

[symbol]

indicates data missing or illegible when filed

From each of the sequences, PCR primers which can acquire a PCR product having a length of about 100 to about 250 bp, and PCR primers which can acquire a PCR product having a length of about 500 to about 600 bp, with the same forward primer, were created. PCR using said primers was performed using the genomic DNA prepared from an EF-2001 strain and a comparative bacterial strain as a template, thereby selecting a primer set which can acquire a PCR product of interest in only the EF-2001 strain and cannot acquire the PCR product even when the DNA of another bacterial strain is used as a template.

### Regarding the Primer Set of the Present Invention

In one aspect of the present invention, the primer set of the present invention can comprise the following:
a pair of an oligonucleotide comprising the base sequence of SEQ ID NO: 1 and an oligonucleotide comprising the base sequence of SEQ ID NO: 2 or 3;
a pair of an oligonucleotide comprising the base sequence of SEQ ID NO: 4 and an oligonucleotide comprising the base sequence of SEQ ID NO: 5 or 6;
a pair of an oligonucleotide comprising the base sequence of SEQ ID NO: 7 and an oligonucleotide comprising the base sequence of SEQ ID NO: 8 or 9;
a pair of an oligonucleotide comprising the base sequence of SEQ ID NO: 10 and an oligonucleotide comprising the base sequence of SEQ ID NO: 11 or 12;
a pair of an oligonucleotide comprising the base sequence of SEQ ID NO: 13 and an oligonucleotide comprising the base sequence of SEQ ID NO: 14 or 15;
a pair of an oligonucleotide comprising the base sequence of SEQ ID NO: 16 and an oligonucleotide comprising the base sequence of SEQ ID NO: 17 or 18;
a pair of an oligonucleotide comprising the base sequence of SEQ ID NO: 19 and an oligonucleotide comprising the base sequence of SEQ ID NO: 20 or 21;
a pair of an oligonucleotide comprising the base sequence of SEQ ID NO: 22 and an oligonucleotide comprising the base sequence of SEQ ID NO: 23 or 24;
a pair of an oligonucleotide comprising the base sequence of SEQ ID NO: 25 and an oligonucleotide comprising the base sequence of SEQ ID NO: 26 or 27;
a pair of an oligonucleotide comprising the base sequence of SEQ ID NO: 28 and an oligonucleotide comprising the base sequence of SEQ ID NO: 29 or 30.

In another aspect of the present invention, the primer set of the present invention can comprise the following:
(a) a primer set (1) for amplifying a part of the base sequence (contig 1) of SEQ ID NO: 51 (contig 1 long):

a forward primer
(SEQ ID NO: 1)
CGAAAAGGATGTAGTCAGCGG;
and
a reverse primer
(SEQ ID NO: 2)
CCGAAGGCGAAACAGAGGAT,

(b) a primer set (2) for amplifying a part of the base sequence (contig 1) of SEQ ID NO: 51 (contig 1 short):

a forward primer
(SEQ ID NO: 1)
CGAAAAGGATGTAGTCAGCGG;
and
a reverse primer
(SEQ ID NO: 3)
CCCAGACATAATCGCATGGC,

(c) a primer set (3) for amplifying a part of the base sequence (contig 4) of SEQ ID NO: 52 (contig 4 long):

a forward primer
(SEQ ID NO: 4)
GCGGCTGCACAATTTATTGC;
and
a reverse primer
(SEQ ID NO: 5)
AGAATACTTGGGCGGTCGTG,

(d) a primer set (4) for amplifying a part of the base sequence (contig 4) of SEQ ID NO: 52 (contig 4 short):

a forward primer
(SEQ ID NO: 6)
GCGGCTGCACAATTTATTGC,
and
a reverse primer
(SEQ ID NO: 4)
AATTCAGCTTCGCTAGATAAGGC;

(e) a primer set (5) for amplifying a part of the base sequence (contig 7) of SEQ ID NO: 53 (contig 7 long):

a forward primer
(SEQ ID NO: 7)
CGCGTATGACTTGCAATCGA;
and
a reverse primer
(SEQ ID NO: 8)
AGGATTGTTTGACGGTGCAA,

(f) a primer set (6) for amplifying a part of the base sequence (contig 7) of SEQ ID NO: 53 (contig 7 short):

a forward primer
(SEQ ID NO: 7)
CGCGTATGACTTGCAATCGA;
and
a reverse primer
(SEQ ID NO: 9)
ACATGAGATAGTTGGGGTAGACA,

(g) a primer set (7) for amplifying a part of the base sequence (contig 11) of SEQ ID NO: 54 (contig 11 long):

a forward primer
(SEQ ID NO: 10)
CTTCAGAGAGCTGGGCGAAG;
and
a reverse primer
(SEQ ID NO: 11)
TACTTTTTAGCTGCCCGCCC,

(h) a primer set (8) for amplifying a part of the base sequence (contig 11) of SEQ ID NO: 54 (contig 11 short):

a forward primer
(SEQ ID NO: 10)
CTTCAGAGAGCTGGGCGAAG;
and
a reverse primer
(SEQ ID NO: 12)
GGGTTGTAGCCCTACCCGAT,

(i) a primer set (9) for amplifying a part of the base sequence (contig 13) of SEQ ID NO: 55 (contig 13 long):

a forward primer
(SEQ ID NO: 13)
CGTAACGTGACATTGCGGAC;
and
a reverse primer
(SEQ ID NO: 14)
ATGCCAGTACGTCGCGTTAA,

(j) a primer set (10) for amplifying a part of the base sequence (contig 13) of SEQ ID NO: 55 (contig 13 short):

a forward primer
(SEQ ID NO: 13)
CGTAACGTGACATTGCGGAC;
and
a reverse primer
(SEQ ID NO: 15)
CGATTGTCAACTAATTGTGCCGA,

(k) a primer set (11) for amplifying a part of the base sequence (contig 16) of SEQ ID NO: 56 (contig 16 long):

a forward primer
(SEQ ID NO: 16)
CATGGCTTGCCGTTTCACAA;
and
a reverse primer
(SEQ ID NO: 17)
ACCGCAACAACTACATACTACCA,

(l) a primer set (12) for amplifying a part of the base sequence (contig 16) of SEQ ID NO: 56 (contig 16 short):

a forward primer
(SEQ ID NO: 16)
CATGGCTTGCCGTTTCACAA;
and
a reverse primer
(SEQ ID NO: 18)
ACCAAAAGGAACGCTACCAGT,

(m) a primer set (13) for amplifying a part of the base sequence (contig 22) of SEQ ID NO: 57 (contig 22 long):

a forward primer
(SEQ ID NO: 19)
TCAGCATAATCCCCAGACGT;
and
a reverse primer
(SEQ ID NO: 20)
AATGAACGCCCTTCAGCAGA,

(n) a primer set (14) for amplifying a part of the base sequence (contig 22) of SEQ ID NO: 57 (contig 22 short):

a forward primer
(SEQ ID NO: 19)
TCAGCATAATCCCCAGACGT;
and
a reverse primer
(SEQ ID NO: 21)
GGCTCCTCTACCTGAACAAACT,

(o) a primer set (15) for amplifying a part of the base sequence (contig 25) of SEQ ID NO: 58 (contig 25 long):

a forward primer
(SEQ ID NO: 22)
GCGTTCAAACTGTTCTGGTGT;
and
a reverse primer
(SEQ ID NO: 23)
TACAAGGCTTGCGAGGTAGC,

(p) a primer set (16) for amplifying a part of the base sequence (contig 25) of SEQ ID NO: 58 (contig 25 short):

a forward primer
(SEQ ID NO: 22)
GCGTTCAAACTGTTCTGGTGT;
and
a reverse primer
(SEQ ID NO: 24)
GCTGCAATGGAAAGCAAATCG,

(q) a primer set (17) for amplifying a part of the base sequence (contig 31) of SEQ ID NO: 59 (contig 31 long):

a forward primer
(SEQ ID NO: 25)
AGGCATATGGGTCATCTGCT;
and
a reverse primer
(SEQ ID NO: 26)
GAGCATCACAGAGCCTCGAA,

(r) a primer set (18) for amplifying a part of the base sequence (contig 31) of SEQ ID NO: 59 (contig 31 short):

a forward primer
(SEQ ID NO: 25)
AGGCATATGGGTCATCTGCT;
and
a reverse primer
(SEQ ID NO: 27)
AGAGATTTTTCAGTATTGCTGGGT,

(s) a primer set (19) for amplifying a part of the base sequence (contig 43) of SEQ ID NO: 60 (contig 43 long):
   a forward primer CGTTGGGTGTGCAGAAATGG (SEQ ID NO: 28); and
   a reverse primer TGTACCGTCAACCTCGTTCG (SEQ ID NO: 29), or
(t) a primer set (20) for amplifying a part of the base sequence (contig 43) of SEQ ID NO: 60 (contig 43 short):

a forward primer
(SEQ ID NO: 28)
CGTTGGGTGTGCAGAAATGG;
and
a reverse primer
(SEQ ID NO: 30)
AACGGGTTGCGACTCTTTTT.

Further, a primer set which can be used in the detection method of the present invention also includes a primer set consisting of an oligonucleotide having a base sequence that is substantially homologous to the base sequences of SEQ ID NOs: 1 to 30 depending on the base length, the PCR condition and the like. In this regard, a sequence which is substantially homologous means that the sequence has a fragment length and homology to the extent that it can function as a primer for PCR or the like, and the sequence preferably has about 90% or greater sequence identity.

For example, the primer set of the present invention does not necessarily need to have 100% homology to the base sequences of SEQ ID NOs: 1 to 30 depending on the purpose of use and the condition. A few bases may be different near the 5′ terminal of a primer for a region of interest. Even when such a primer is used, it is possible to appropriately amplify a DNA fragment of interest by considering the annealing temperature or the like.

For example, when high specificity is required in detecting a lactic acid bacterium EF-2001 strain, a fully homologous sequence site (the base sequences of SEQ ID NOs: 1 to 30) should be used, and a PCR condition under which annealing occurs only with such a sequence should be selected. On the other hand, when a condition with relatively low specificity is allowed, a sequence which is different from the base sequences of SEQ ID NOs: 1 to 30 in a few bases (e.g., a sequence having about 90% or greater sequence identity) can be used, and a PCR condition under which annealing occurs even with such a sequence can be selected.

The primer set of the present invention can be synthesized by a common DNA synthesizing method that is well known to those skilled in the art, e.g., a DNA synthesizer. The primer of the present invention can also be obtained by entrusting synthesis to a DNA synthesis operator.

A lactic acid bacterium EF-2001 strain in a sample can be detected by using the primer set of the present invention to perform PCR using the chromosomal DNA of a test bacterium contained in the sample as a template and determining the presence or absence of an amplification product. Specifically, when a reaction of PCR specific to the sequence of the primers occurs, a region (target region) sandwiched by the sequences corresponding to the sequence of each of the primers that are a pair in the chromosomal DNA in the lactic acid bacterium EF-2001 strain is amplified.

Thus, when an amplification product is obtained using the primer set of the present invention, the test bacterium is identified to be a lactic acid bacterium EF-2001 strain, or the test bacterium is determined to at least comprise a lactic acid bacterium EF-2001 strain.

Further, when an amplification product is obtained, determination of the presence or absence of the amplification product may comprise determination of the length of the amplification product. For example, when a primer set for detecting a lactic acid bacterium EF-2001 strain which comprises oligonucleotides consisting of each of the combinations of SEQ ID NO: 1 and SEQ ID NO: 2, SEQ ID NOs: 4 and 5, SEQ ID NOs: 7 and 8, SEQ ID NOs: 10 and 11, SEQ ID NOs: 13 and 14, SEQ ID NOs: 16 and 17, SEQ ID NOs: 19 and 20, SEQ ID NOs: 22 and 23, SEQ ID NOs: 25 and 26, or SEQ ID NOs: 28 and 29 is used, an amplification product of about 500 to about 600 bp is generally obtained for each of the sequences if the test bacterium comprises a lactic acid bacterium EF-2001 strain. Further, when a primer set for detecting a lactic acid bacterium EF-2001 strain which comprises oligonucleotides consisting of each of the combinations of SEQ ID NO: 1 and SEQ ID NO: 3, SEQ ID NOs: 4 and 6, SEQ ID NOs: 7 and 9, SEQ ID NOs: 10 and 12, SEQ ID NOs: 13 and 15, SEQ ID NOs: 16 and 18, SEQ ID NOs: 19 and 21, SEQ ID NOs: 22 and 24, SEQ ID NOs: 25 and 27, or SEQ ID NOs: 28 and 30 is used, an amplification product of about 100 to about 250 bp is generally obtained for each of the sequences if the test bacterium comprises a lactic acid bacterium EF-2001 strain.

The following amplification products are obtained when the primer set for detecting a lactic acid bacterium EF-2001 strain comprising oligonucleotides of the present invention is used.
SEQ ID NO: 31: an amplification product amplified by a primer set consisting of a pair of an oligonucleotide comprising the base sequence of SEQ ID NO: 1 and an oligonucleotide comprising the base sequence of SEQ ID NO: 2
SEQ ID NO: 32: an amplification product amplified by a primer set consisting of a pair of an oligonucleotide comprising the base sequence of SEQ ID NO: 1 and an oligonucleotide comprising the base sequence of SEQ ID NO: 3
SEQ ID NO: 33: an amplification product amplified by a primer set consisting of a pair of an oligonucleotide comprising the base sequence of SEQ ID NO: 4 and an oligonucleotide comprising the base sequence of SEQ ID NO: 5
SEQ ID NO: 34: an amplification product amplified by a primer set consisting of a pair of an oligonucleotide comprising the base sequence of SEQ ID NO: 4 and an oligonucleotide comprising the base sequence of SEQ ID NO: 6
SEQ ID NO: 35: an amplification product amplified by a primer set consisting of a pair of an oligonucleotide comprising the base sequence of SEQ ID NO: 7 and an oligonucleotide comprising the base sequence of SEQ ID NO: 8
SEQ ID NO: 36: an amplification product amplified by a primer set consisting of a pair of an oligonucleotide comprising the base sequence of SEQ ID NO: 7 and an oligonucleotide comprising the base sequence of SEQ ID NO: 9
SEQ ID NO: 37: an amplification product amplified by a primer set consisting of a pair of an oligonucleotide comprising the base sequence of SEQ ID NO: 10 and an oligonucleotide comprising the base sequence of SEQ ID NO: 11
SEQ ID NO: 38: an amplification product amplified by a primer set consisting of a pair of an oligonucleotide comprising the base sequence of SEQ ID NO: 10 and an oligonucleotide comprising the base sequence of SEQ ID NO: 12
SEQ ID NO: 39: an amplification product amplified by a primer set consisting of a pair of an oligonucleotide comprising the base sequence of SEQ ID NO: 13 and an oligonucleotide comprising the base sequence of SEQ ID NO: 14
SEQ ID NO: 40: an amplification product amplified by a primer set consisting of a pair of an oligonucleotide comprising the base sequence of SEQ ID NO: 13 and an oligonucleotide comprising the base sequence of SEQ ID NO: 15
SEQ ID NO: 41: an amplification product amplified by a primer set consisting of a pair of an oligonucleotide comprising the base sequence of SEQ ID NO: 16 and an oligonucleotide comprising the base sequence of SEQ ID NO: 17
SEQ ID NO: 42: an amplification product amplified by a primer set consisting of a pair of an oligonucleotide comprising the base sequence of SEQ ID NO: 16 and an oligonucleotide comprising the base sequence of SEQ ID NO: 18
SEQ ID NO: 43: an amplification product amplified by a primer set consisting of a pair of an oligonucleotide comprising the base sequence of SEQ ID NO: 19 and an oligonucleotide comprising the base sequence of SEQ ID NO: 20
SEQ ID NO: 44: an amplification product amplified by a primer set consisting of a pair of an oligonucleotide comprising the base sequence of SEQ ID NO: 19 and an oligonucleotide comprising the base sequence of SEQ ID NO: 21
SEQ ID NO: 45: an amplification product amplified by a primer set consisting of a pair of an oligonucleotide comprising the base sequence of SEQ ID NO: 22 and an oligonucleotide comprising the base sequence of SEQ ID NO: 23
SEQ ID NO: 46: an amplification product amplified by a primer set consisting of a pair of an oligonucleotide comprising the base sequence of SEQ ID NO: 22 and an oligonucleotide comprising the base sequence of SEQ ID NO: 24
SEQ ID NO: 47: an amplification product amplified by a primer set consisting of a pair of an oligonucleotide comprising the base sequence of SEQ ID NO: 25 and an oligonucleotide comprising the base sequence of SEQ ID NO: 26
SEQ ID NO: 48: an amplification product amplified by a primer set consisting of a pair of an oligonucleotide comprising the base sequence of SEQ ID NO: 25 and an oligonucleotide comprising the base sequence of SEQ ID NO: 27
SEQ ID NO: 49: an amplification product amplified by a primer set consisting of a pair of an oligonucleotide comprising the base sequence of SEQ ID NO: 28 and an oligonucleotide comprising the base sequence of SEQ ID NO: 29
SEQ ID NO: 50: an amplification product amplified by a primer set consisting of a pair of an oligonucleotide comprising the base sequence of SEQ ID NO: 28 and an oligonucleotide comprising the base sequence of SEQ ID NO: 30

Further, each of the base sequences of SEQ ID NOs: 31 to 50 is a part of the base sequence of any of SEQ ID NOs: 51 to 60.

Each primer sequence of SEQ ID NOs: 1 to 30, and the base sequences of SEQ ID NOs: 31 to 50 and the base sequences of SEQ ID NOs: 51 to 60 are sequences specific to a lactic acid bacterium EF-2001 strain. Thus, it is also possible to perform FISH method, Southern hybridization, dot hybridization or the like using a part of these sequences as a DNA probe.

In the present invention, detection of a lactic acid bacterium EF-2001 strain comprises detecting whether or not a lactic acid bacterium EF-2001 strain is present in a sample, and when a test bacterium is a single species, detection of a lactic acid bacterium EF-2001 strain comprises identifying whether or not the test bacterium is a lactic acid bacterium EF-2001 strain.

In one embodiment of the present invention, any sample can be used for detection of a lactic acid bacterium EF-2001 strain as long as a lactic acid bacterium EF-2001 strain is present or may be present in the sample. Examples of the sample can include a mixed culture of lactic acid bacteria, fermented food comprising a microorganism such as miso, soy sauce, alcohol such as wine, pickles, yoghurt, or cheese, feces of experimental animals such as mice or rats or humans, bacteria that are present in an environment such as soil, and the like. A test bacterium may be an isolated single species, or may be a mixture comprising a plurality of bacterial species.

In one embodiment of the present invention, when a dead bacterium is used as a test bacterium, DNA may be directly prepared from a sample and used for PCR. When a live bacterium is used, a test bacterial cell may be isolated from a sample by colony isolation or the like, and DNA may be prepared from the test bacterial cell and subjected to PCR. Methods for extracting a nucleic acid from these samples are not particularly limited. For example, it is possible to use a highly versatile kit, such as Kaneka Easy DNA Extraction Kit version 2, which is capable of extracting a nucleic acid from a cultured cell including an animal cell, a plant cell, a microorganism and the like, or use a commercially available extraction kit (such as DNeasy Blood & Tissue kit (QIAGEN) or Isogen) as commonly used for preparation of a genomic DNA of a lactic acid bacterium after degrading a cell wall by an enzyme such as Lysozyme or physically destroying a cell wall by a bead or the like.

### Regarding the Detection Method of the Present Invention

In one aspect of the present invention, the method for detecting a lactic acid bacterium EF-2001 strain of the present invention can comprise (1) the step of amplifying a nucleic acid fragment using the primer set of the present invention and (2) the step of detecting a nucleic acid fragment obtained in the step (1).

Specific examples of the detection method of the present invention can include PCR method, FISH method, Southern hybridization, dot hybridization and the like. PCR method is particularly preferable. Among these methods, a method for detecting a lactic acid bacterium EF-2001 strain using PCR method is not particularly limited. Apart from the use of the primer set of the present invention, commonly used PCR reagents and apparatus such as DNA polymerase can also be used.

In one embodiment, a DNA polymerase which is used for PCR is not particularly limited, but is preferably, for example, KOD FX Neo (TOYOBO), ExTaq (Takara Bio), KOD DNA polymerase (TOYOBO), KOD-plus-polymerase (TOYOBO) or the like.

In one embodiment, a PCR reaction condition can be appropriately set depending on the optimal temperature for a DNA polymerase that is used, the length or the type of DNA to be synthesized, or the like. A condition under which 20 to 40 cycles of a cycle of “5 to 30 seconds at 90 to 98° C. (thermal denaturation/dissociation)→5 to 30 seconds at 55 to 60° C. (annealing)→30 to 60 seconds at 65 to 80° C. (synthesis/extension) “are performed in total can be set as a cycle condition. For example, 25 cycles of “10 seconds at 98° C.→30 seconds at 60° C.→30 seconds at 68° C.” can also be performed. In this case, a temperature which was used for design of a primer can be used as the annealing temperature. For example, when KOD FX Neo is used as a polymerase, the reaction can be configured as two-step reaction which omits the annealing temperature depending on the primer size (e.g., 20 mer or greater). Regarding the quantity ratio of a template DNA to a primer, for example, in the case of a live bacterium, 0.3 μM of each primer can be used for 200 ng of the template DNA. In the case of a dead bacterium, a band in electrophoresis can be made clearer by increasing the template DNA depending on the situation of thermal treatment.

The presence or absence or the size of an amplification product obtained by PCR can be determined by a common nucleic acid detection method. For example, an amplification product can be detected by electrophoresis by agarose gel electrophoresis, followed by staining with ethidium bromide or SYBR Green I. Further, the amount of an amplification product can be determined by fluorescence intensity, and the molecular weight of an amplification product can be determined by comparison with a molecular weight marker. The presence or absence of an amplification product can also be confirmed by subjecting a PCR product to cycle sequencing, followed by measuring the base sequence and the length using a DNA sequencer. Further, real-time PCR method enables detection of an amplification reaction over time.

In one aspect of the present invention, the primer set of the present invention can be configured to be a kit for detecting a lactic acid bacterium EF-2001 strain in combination with other elements. Examples of other elements include extraction of a nucleic acid, PCR, and any one or more types of reagents necessary for detection of an amplification product. This kit for detecting a lactic acid bacterium EF-2001 strain may comprise a DNA fragment which has a part of the sequence of the chromosomal DNA of the lactic acid bacterium EF-2001 strain and can be amplified by the primer set of the present invention as a positive control, and/or a DNA fragment having a base sequence which corresponds to the primer set of the present invention but has a mismatch in one base or a few bases as a negative control.

The primer set of the present invention can be used for detection of a lactic acid bacterium EF-2001 strain as described above. Further, when industrially manufacturing a lactic acid bacterium EF-2001 strain bacterial cell or food and drink or the like comprising the bacterial cell, use of the primer set of the present invention facilitates measurement of the number of bacteria and control of the fermentation state.

### Regarding the Lactic Acid Bacterium Composition of the Present Invention

In one aspect of the present invention, the lactic acid bacterium composition of the present invention can comprise a heat-treated lactic acid bacterium product which can be obtained by heating a live bacterium of a lactic acid bacterium Enterococcus faecalis EF-2001 strain. In one embodiment, the lactic acid bacterium composition of the present invention preferably comprises a nucleic acid fragment which is not amplified when any one or more of primer sets for detecting a lactic acid bacterium EF-2001 strain comprising oligonucleotides consisting of each of the combinations of SEQ ID NO: 1 and SEQ ID NO: 2, SEQ ID NOs: 4 and 5, SEQ ID NOs: 7 and 8, SEQ ID NOs: 10 and 11, SEQ ID NOs: 13 and 14, SEQ ID NOs: 16 and 17, SEQ ID NOs: 19 and 20, SEQ ID NOs: 22 and 23, SEQ ID NOs: 25 and 26, and/or SEQ ID NOs: 28 and 29 are used, and which is amplified when any one or more of primer sets for detecting a lactic acid bacterium EF-2001 strain comprising oligonucleotides consisting of each of the combinations of SEQ ID NO: 1 and SEQ ID NO: 3, SEQ ID NOs: 4 and 6, SEQ ID NOs: 7 and 9, SEQ ID NOs: 10 and 12, SEQ ID NOs: 13 and 15, SEQ ID NOs: 16 and 18, SEQ ID NOs: 19 and 21, SEQ ID NOs: 22 and 24, SEQ ID NOs: 25 and 27, or SEQ ID NOs: 28 and 30 are used.

As used herein, “a nucleic acid fragment not being amplified” or “a band disappearing” refers to an amplification efficiency or an amplification amount being less than about 30% in comparison to those of a nucleic acid fragment derived from an untreated (e.g., without heat treatment) lactic acid bacterium EF-2001 strain when a nucleic acid fragment of a lactic acid bacterium EF-2001 strain is amplified by using the primer set for detecting a lactic acid bacterium EF-2001 strain of the present invention.

In one embodiment, “a nucleic acid fragment not being amplified” or “a band disappearing” refers to an amplification efficiency or an amplification amount being less than about 20%, preferably less than about 10%, or further preferably less than about 5% in comparison to those of a nucleic acid fragment derived from an untreated (e.g., without heat treatment) lactic acid bacterium EF-2001 strain when a nucleic acid fragment of a lactic acid bacterium EF-2001 strain is amplified by using the primer set for detecting a lactic acid bacterium EF-2001 strain of the present invention.

In one embodiment, the lactic acid bacterium composition of the present invention can be heated to be a dead bacterial cell and then dried by lyophilization, spray drying, or a drum dryer to be a powdery dry bacterial cell. Safety and preservability can be thereby improved, and said composition can be utilized for various food. Heating of a lactic acid bacterium EF-2001 strain can be performed for any duration of time and at any temperature which can result in a dead bacterial cell. Heating can be performed, for example, for about 5 minutes or longer, about 10 minutes or longer, about 15 minutes or longer, about 20 minutes or longer, about 25 minutes or longer, about 30 minutes or longer, about 45 minutes or longer, or about 60 minutes or longer, for example, at a temperature of about 50° C. or greater, about 60° C. or greater, about 70° C. or greater, about 80° C. or greater, about 90° C. or greater, about 100° C. or greater, about 110° C. or greater, or about 120° C. or greater or the like. Heating may be performed with any combination of temperature and duration of time as long as a sterilized or dead bacterial cell can be obtained by heating.

In one embodiment of the present invention, when obtaining the lactic acid bacterium composition of the present invention, a lactic acid bacterium Enterococcus faecalis EF-2001 strain is heated at the above-described temperature and for the above-described duration of time to obtain a plurality of lots of a heat-treated lactic acid bacterium product, and a nucleic acid fragment is extracted from these heat-treated lactic acid bacterium products. The nucleic acid fragment is then amplified using at least one of the primer sets of the present invention described in other portions herein. A lot of the heat-treated lactic acid bacterium product in which the amplified nucleic acid fragment has been detected is then selected, and the heat-treated lactic acid bacterium product contained in the lot can be made as the lactic acid bacterium composition of the present invention. An apparatus or an approach commonly used in the art can be employed for heating and nucleic acid amplification. For example, an apparatus or an approach which can be used in the detection method described in other portions herein can be employed. When heating a bacterial cell, a class-I pressure vessel equipped with a stirring apparatus can be commonly used, and a class-II pressure vessel can also be used depending on the temperature. A small pressure vessel or the like can also be used if the liquid amount is small. In other embodiments, a continuous sterilizing apparatus utilizing a plate or a tube can also be used as a heat conductor.

In one embodiment of the present invention, the TNF-α production amount of the lactic acid bacterium composition of the present invention can be increased by heating and stabilizing the composition. The lactic acid bacterium composition of the present invention preferably comprises a nucleic acid fragment which is not amplified when any one or more of primer sets for detecting a lactic acid bacterium EF-2001 strain comprising oligonucleotides consisting of each of the combinations of SEQ ID NO: 1 and SEQ ID NO: 2, SEQ ID NOs: 4 and 5, SEQ ID NOs: 7 and 8, SEQ ID NOs: 10 and 11, SEQ ID NOs: 13 and 14, SEQ ID NOs: 16 and 17, SEQ ID NOs: 19 and 20, SEQ ID NOs: 22 and 23, SEQ ID NOs: 25 and 26, and/or SEQ ID NOs: 28 and 29 are used, and which is amplified when any one or more of primer sets for detecting a lactic acid bacterium EF-2001 strain comprising oligonucleotides consisting of each of the combinations of SEQ ID NO: 1 and SEQ ID NO: 3, SEQ ID NOs: 4 and 6, SEQ ID NOs: 7 and 9, SEQ ID NOs: 10 and 12, SEQ ID NOs: 13 and 15, SEQ ID NOs: 16 and 18, SEQ ID NOs: 19 and 21, SEQ ID NOs: 22 and 24, SEQ ID NOs: 25 and 27, and/or SEQ ID NOs: 28 and 30 are used.

In one embodiment, it is possible to confirm whether or not the TNF-α production amount of the lactic acid bacterium composition of the present invention is increased by using a band of a primer set for detecting a long sequence disappearing and a band of a primer set for detecting a short sequence remaining as an indicator. For example, by using a nucleic acid fragment not being amplified by a primer set (long) of SEQ ID NOs: 16 and 17 amplifying a part of the base sequence of contig 16 and the nucleic acid fragment being amplified by a primer set (short) of SEQ ID NOs: 16 and 18 as an indicator, it is possible to determine that the lactic acid bacterium composition of the present invention subjected to heat treatment showing such an amplification result has an increased TNF-α production amount.

In one embodiment of the present invention, any diluent can be mixed into the lactic acid bacterium composition of the present invention to prevent individual bacterial cells which are placed in an isolated state from being thereby aggregated when each bacterial cell is added to food or the like and absorbs water. Preferably, a diluent is not only fine powdery but also fluidic as highly as possible in order to facilitate homogeneous mixing with bacterial cells. Given the safety upon addition to food, the effect on a flavor or a property of food and the like, a diluent is preferably a polysaccharide or a protein. Examples of the polysaccharide can include naturally-occurring polysaccharides such as lactose, polydextrose, cyclodextrin, corn starch, microcrystalline cellulose, starch, or galactomannan, enzyme-treated hydrolyzate thereof and the like, and examples of the protein can include soy bean protein, casein, wheat gluten, egg white albumin and the like. It is also possible to use any two or more types thereof in combination.

As used herein, “or” is used when “at least one or more” of the matters listed in the sentence can be employed. When explicitly described herein as “within the range” of “two values”, the range also includes the two values themselves.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. The present disclosure is described below based on Examples. The aforementioned description and the following Examples are not provided to limit the present disclosure, but for the sole purpose of exemplification. Thus, the scope of the present disclosure is not limited to the embodiments and Examples specifically described herein and is limited only by the scope of claims.

### EXAMPLES

### Example 1: Detection 1 of a Lactic Acid Bacterium EF-2001 Strain (Comparison with Enterococcus faecalis Subspecies)

### (1) Test Bacterial Strain

(A) A lactic acid bacterium EF-2001 strain, and (B) E. faecalis NBRC3971, (C) E. faecalis NBRC3989, (D) E. faecalis NBRC12970, (E) E. faecalis NBRC100480, (F) E. faecalis NBRC100482, (G) E. faecalis NBRC100483 and (H) E. faecalis NBRC100484, which are Enterococcus faecalis subspecies, were used as test bacterial strains.

### (2) Preparation of a Lactic Acid Bacterium DNA

Buffer comprising an enzyme such as lysozyme was added to the above bacterial cells collected from culture liquid of each bacterial cell, and the cell membrane was degraded. After treatment with proteinase and RNase, sodium acetate was added and a precipitate precipitated with ethanol was rinsed with ethanol, followed by drying the product thereof. Buffer such as TE was added thereto and dissolved to prepare solution, which was used as DNA solution for PCR. DNA solution can also be prepared using a commercially available DNA extracting kit.

### (3) PCR Reaction

The DNA solution of the test bacterial strains obtained in the above (2) was used as a template, and a primer set consisting of a pair of oligonucleotides comprising the base sequences of SEQ ID NO: 1 and SEQ ID NO: 2 (contig 1), SEQ ID NOs: 4 and 5 (contig 4), SEQ ID NOs: 7 and 8 (contig 7), SEQ ID NOs: 10 and 11 (contig 11), SEQ ID NOs: 13 and 14 (contig 13), SEQ ID NOs: 16 and 17 (contig 16), SEQ ID NOs: 19 and 20 (contig 22), SEQ ID NOs: 22 and 23 (contig 25), SEQ ID NOs: 25 and 26 (contig 31), and SEQ ID NOs: 28 and 29 (contig 43) and a primer set consisting of a pair of oligonucleotides comprising the base sequences of SEQ ID NO: 1 and SEQ ID NO: 3 (contig 1), SEQ ID NOs: 4 and 6 (contig 4), SEQ ID NOs: 7 and 9 (contig 7), SEQ ID NOs: 10 and 12 (contig 11), SEQ ID NOs: 13 and 15 (contig 13), SEQ ID NOs: 16 and 18 (contig 16), SEQ ID NOs: 19 and 21 (contig 22), SEQ ID NOs: 22 and 24 (contig 25), SEQ ID NOs: 25 and 27 (contig 31), and SEQ ID NOs: 28 and 30 (contig 43) were used to perform a PCR reaction according to the method of KOD FX Neo (TOYOBO), which is a PCR reaction reagent kit. The total amount of PCR reaction liquid was 50 μL, and 25 cycles of a cycle of preheating reaction liquid comprising 25.0 μL of 2×PCR buffer accompanying the kit, 10.0 μL of 2 mM dNTPs, 1.0 U of DNA polymerase, 0.2 μL of primer mixture, and 10 to 200 ng of the template DNA (in the case of a live bacterium) or 10 to 400 ng of the template DNA (in the case of a dear bacterium) for 10 seconds at 94° C. followed by denaturation for 10 seconds at 98° C., annealing for 30 seconds at 60° C., and extension for 30 seconds at 68° C. were performed using Thermal cycler GenAtlas (ASTEC).

As a positive control, primers (F: SEQ ID NO: 61 and R: SEQ ID NO: 62) which were designed so that the amplification product would be 200 to 300 bp were used, with the 16S rDNA of a lactic acid bacterium EF-2001 strain as a template.

### (4) Agarose Electrophoresis

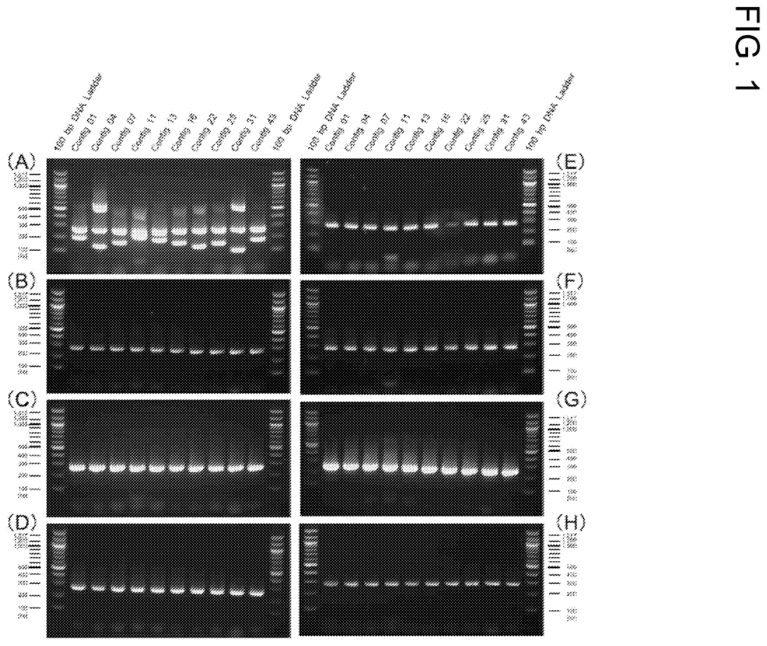

The PCR product obtained by the PCR reaction was subjected to electrophoresis for 30 minutes at 100 V with 1.4% agarose gel comprising GelRed Nucleic Acid Gel Stain (FUJIFILN Co., Ltd.). Next, the band showing amplification of the PCR product was observed with WSE-5400-UP Printgraph Classic (ATTO Co., Ltd.). FIG. 1 shows the results.

As shown FIG. 1, a band of the positive control of about 284 bp was observed in all subspecies of Enterococcus faecalis which were used in this test. A band of about 100 to about 250 bp and a band of about 500 to about 600 bp were observed only when the DNA of the EF-2001 strain was used (FIG. 1(A)), and it was confirmed that the primer set of the present invention can specifically detect only a lactic acid bacterium EF-2001 strain from Enterococcus faecalis subspecies.

### Example 2: Detection 2 of a Lactic Acid Bacterium EF-2001 Strain (Comparison with Other Lactic Acid Bacteria)

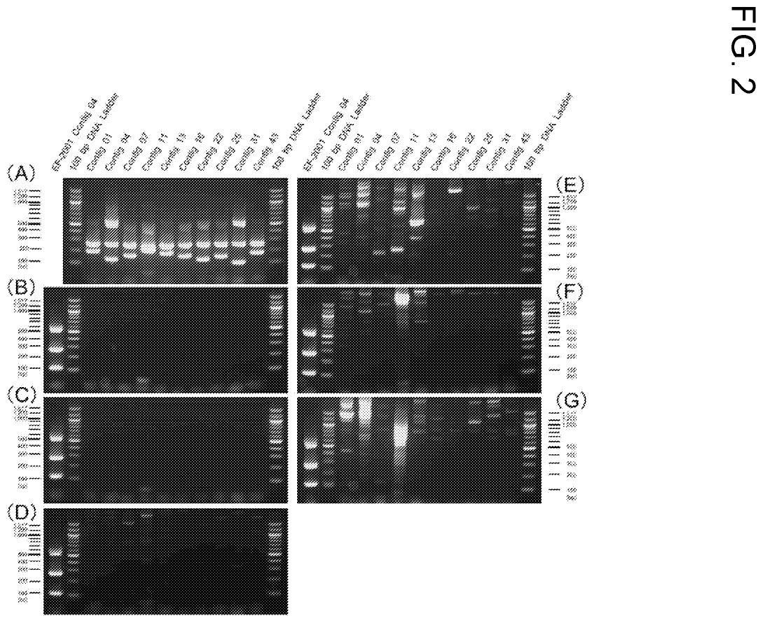

All steps were performed in the same manner as Example 1 except that (A) a lactic acid bacterium EF-2001 strain, and (B) E. faecium Aus0004, (C) E. gallinarum LMG13129, (D) Lactobacillus rhamnosus NBRC3425, (E) L. plantarum NBRC15891, (F) L. paracasei NBRC15906, and (G) L. lactis NBRC102622, which are other lactic acid bacteria, were used as test bacterial strains. FIG. 2 shows the results.

As shown in FIG. 2, a band of the positive control of about 284 bp, a band of about 100 to about 250 bp, and a band of about 500 to about 600 bp were observed only when the DNA of the lactic acid bacterium EF-2001 strain was used (FIG. 2(A)). No band was observed in the lactic acid bacteria other than the lactic acid bacterium EF-2001 strain. It was confirmed from these results that the primer set of the present invention can specifically detect only a lactic acid bacterium EF-2001 strain from other lactic acid bacteria.

### Example 3: Detection 3 of a Lactic Acid Bacterium EF-2001 Strain (Live Bacterium and Heated Bacterium)

### (1) Test Bacterial Strain

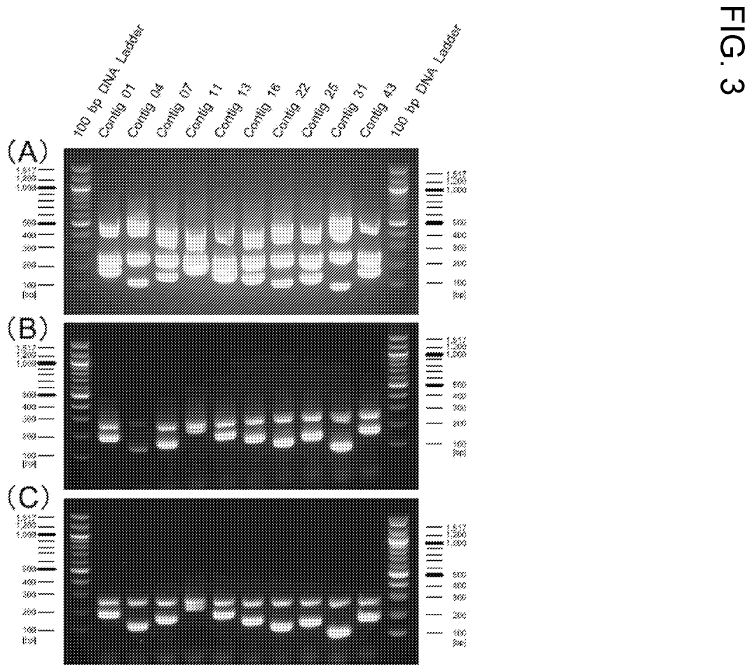

A lactic acid bacterium EF-2001 strain was used as a test bacterial strain, and a nucleic acid was detected using the DNA extracted from three types of bacterial cells, a live bacterium (FIG. 3(A) culture liquid), a dead bacterium (FIG. 3(B) liquid after heat treatment), and a dead bacterium (FIG. 3(C) product bulk) as a template.

(2) The test was performed using the same DNA preparation, PCR condition, and PCR primers as those of Examples 1 and 2. FIG. 3 shows the results of performing agarose electrophoresis under the same condition as that of Examples 1 and 2.

As shown in FIG. 3, a band of about 100 to about 250 bp was observed for both the live bacterium and the dead bacterium of the lactic acid bacterium EF-2001 strain. It was revealed that the primer set of the present invention exhibits a specific detection ability on not only a live bacterium of a lactic acid bacterium EF-2001 strain but also on a heat-treated bacterium (dead bacterium). These results mean that a lactic acid bacterium EF-2001 strain can be detected in a product or the like utilizing a heat-treated lactic acid bacterium EF-2001 strain, let alone a product or the like utilizing a live bacterium of a lactic acid bacterium EF-2001 strain, which is very useful.

### Example 4: Disappearance of a Band Due to Heating

Disappearance of a band of the primer set of the present invention due to heat treatment was confirmed using a lactic acid bacterium EF-2001 strain as a test bacterial strain. In addition to an untreated bacterium (live bacterium), lactic acid bacterium EF-2001 strains that were heat-treated at 70° C., 90° C., and 110° C. were used.

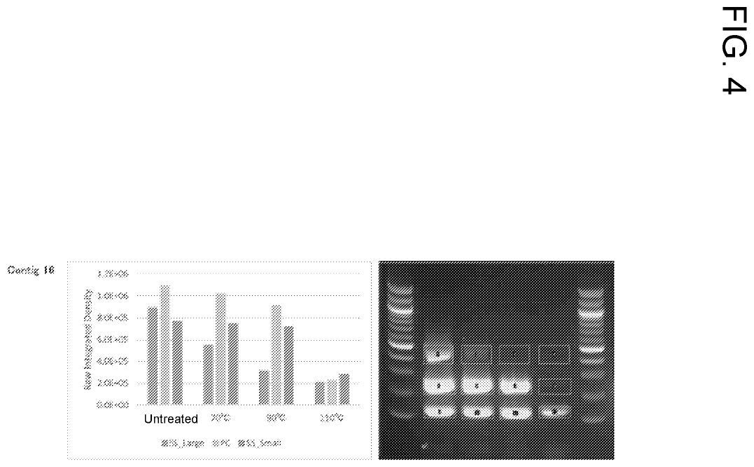

As one example, primers of SEQ ID NOs: 16 and 17 amplifying a part of the base sequence of contig 16, and SEQ ID NOs: 16 and 18 were used to study the amplification ability of a nucleic acid fragment under the same PCR condition and the like as that of Example 1. FIG. 4 shows the results.

As shown in FIG. 4, it was found that a band of the primer set of SEQ ID NOs: 16 and 17 (long) disappeared as the heating temperature was increased. Since a product utilizing a heat-treated lactic acid bacterium EF-2001 strain needs a certain thermal history, disappearance of the band of the primers for detecting a long sequence shows the result meaning that a certain thermal history is being given, which is very useful. Meanwhile, for the primer set of SEQ ID NOs: 16 and 18 (short), a band upon electrophoresis could be observed even when heating was performed at 110° C.

### Example 5: Measurement of the TNF-α Production Amount

The change in the TNF-α production amount of a lactic acid bacterium EF-2001 strain due to heat treatment was confirmed using a lactic acid bacterium EF-2001 strain as a test bacterial strain. An untreated bacterium (live bacterium) and a lactic acid bacterium EF-2001 strain which was heat-treated at 110° C. were used.

First, RAW264.7 cells were used to induce TNF-α. 100 μL of RAW264.7 cell solution adjusted to 1.0×10⁶ cells/mL per 1 well was dispensed into 96 wells, and the solution was cultured for 24 hours in a 5% CO₂ thermostatic vessel at 37° C. The culture supernatant was removed and the cultured solution was washed with an FBS-free RPMI-1640 medium, followed by adding 100 μL of sample solution and reacting the mixture thereof for 6 hours in a 5% CO₂ thermostatic vessel at 37° C. The sample was prepared by preparing 1 mg/mL of EF-2001 solution using FBS-free RPMI-1640 Medium with L-glutamine and sodium bicarbonate, liquid, sterile-filterd, suitable for cell culture (R8758-500ML; SIGMA) and adjusting the solution by appropriately diluting it.

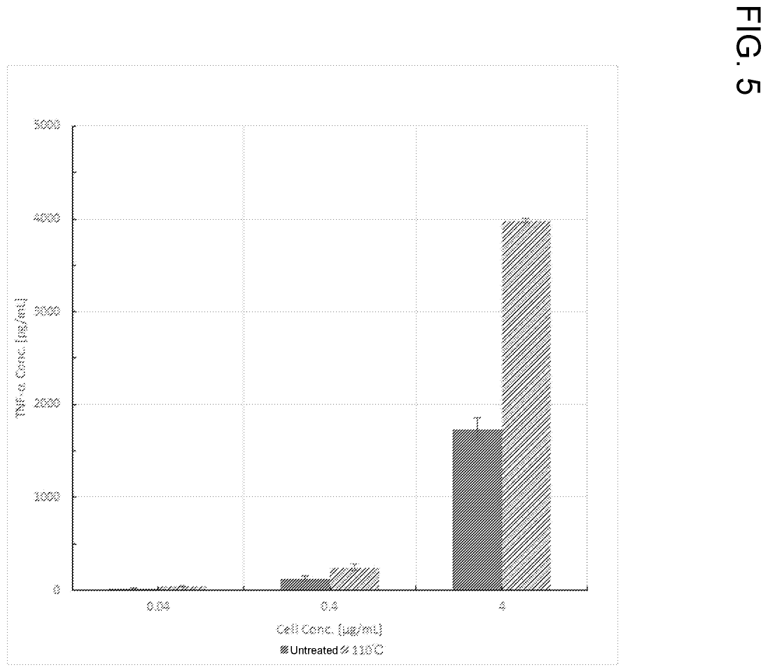

After 6 hours, the supernatant was collected, and the product thereof was stored at −80° C. until the TNF-α measurement. After the sample was appropriately diluted, the concentration of TNF-α which was induced and produced was measured according to the protocol of “Mouse TNF-α Quantikine ELlSA Kit”. FIG. 5 shows the result thereof.

As shown in FIG. 5, it was confirmed that addition of heat treatment (110° C.) to the lactic acid bacterium EF-2001 strain resulted in an increase in the TNF-α production amount, which means that a product utilizing a heat-treated lactic acid bacterium EF-2001 strain has an increased TNF-α production amount. In combination with the results shown in Example 4 that a band of primers for detecting a long sequence disappears and a band of primers for detecting a short sequence remains as a result of giving a certain thermal history, the above result showed a possibility that confirming that a band of primers for detecting a long sequence disappears and a band of primers for detecting a short sequence remains may be an indicator as to whether a lactic acid bacterium EF-2001 strain with an increased TNF-α production amount has been obtained.

### (Note)

As described above, the present disclosure is exemplified by the use of its preferred embodiments. However, it is understood that the scope of the present disclosure should be interpreted solely based on the Claims. It is also understood that any patent, any patent application, and any references cited herein should be incorporated herein by reference in the same manner as the contents are specifically described herein. The present application claims priority to Japanese Patent Application No. 2021-21102 filed on Feb. 12, 2021 with the Japan Patent Office, the content of which being incorporated by reference in the same manner as if the entirety thereof constitutes the content of the present application.

### INDUSTRIAL APPLICABILITY

Since the method of the present invention can rapidly, simply, and specifically detect a lactic acid bacterium Enterococcus faecalis EF-2001 strain, said method can be expected to be widely applied to estimating of a gene involved in various activities of an EF-2001 strain, development of functional food or supplements using the gene, and the like.

### SEQUENCE LISTING FREE TEXT

SEQ ID NO: 1: Forward primer in contig 1
SEQ ID NO: 2: Reverse primer (long) in contig 1
SEQ ID NO: 3: Reverse primer (short) in contig 1
SEQ ID NO: 4: Forward primer in contig 4
SEQ ID NO: 5: Reverse primer (long) in contig 4
SEQ ID NO: 6: Reverse primer (short) in contig 4
SEQ ID NO: 7: Forward primer in contig 7
SEQ ID NO: 8: Reverse primer (long) in contig 7
SEQ ID NO: 9: Reverse primer (short) in contig 7
SEQ ID NO: 10: Forward primer in contig 11
SEQ ID NO: 11: Reverse primer (long) in contig 11
SEQ ID NO: 12: Reverse primer (short) in contig 11
SEQ ID NO: 13: Forward primer in contig 13
SEQ ID NO: 14: Reverse primer (long) in contig 13
SEQ ID NO: 15: Reverse primer (short) in contig 13
SEQ ID NO: 16: Forward primer in contig 16
SEQ ID NO: 17: Reverse primer (long) in contig 16
SEQ ID NO: 18: Reverse primer (short) in contig 16
SEQ ID NO: 19: Forward primer in contig 22
SEQ ID NO: 20: Reverse primer (long) in contig 22
SEQ ID NO: 21: Reverse primer (short) in contig 22
SEQ ID NO: 22: Forward primer in contig 25
SEQ ID NO: 23: Reverse primer (long) in contig 25
SEQ ID NO: 24: Reverse primer (short) in contig 25
SEQ ID NO: 25: Forward primer in contig 31
SEQ ID NO: 26: Reverse primer (long) in contig 31
SEQ ID NO: 27: Reverse primer (short) in contig 31
SEQ ID NO: 28: Forward primer in contig 43
SEQ ID NO: 29: Reverse primer (long) in contig 43
SEQ ID NO: 30: Reverse primer (short) in contig 43
SEQ ID NO: 31: Amplification product (long) of contig 1
SEQ ID NO: 32: Amplification product (short) of contig 1
SEQ ID NO: 33: Amplification product (long) of contig 4
SEQ ID NO: 34: Amplification product (short) of contig 4
SEQ ID NO: 35: Amplification product (long) of contig 7
SEQ ID NO: 36: Amplification product (short) of contig 7
SEQ ID NO: 37: Amplification product (long) of contig 11
SEQ ID NO: 38: Amplification product (short) of contig 11
SEQ ID NO: 39: Amplification product (long) of contig 13
SEQ ID NO: 40: Amplification product (short) of contig 13
SEQ ID NO: 41: Amplification product (long) of contig 16
SEQ ID NO: 42: Amplification product (short) of contig 16
SEQ ID NO: 43: Amplification product (long) of contig 22
SEQ ID NO: 44: Amplification product (short) of contig 22
SEQ ID NO: 45: Amplification product (long) of contig 25
SEQ ID NO: 46: Amplification product (short) of contig 25
SEQ ID NO: 47: Amplification product (long) of contig 31
SEQ ID NO: 48: Amplification product (short) of contig 31
SEQ ID NO: 49: Amplification product (long) of contig 43
SEQ ID NO: 50: Amplification product (short) of contig 43
SEQ ID NO: 51: Contig 1
SEQ ID NO: 52: Contig 4
SEQ ID NO: 53: Contig 7
SEQ ID NO: 54: Contig 11
SEQ ID NO: 55: Contig 13
SEQ ID NO: 56: Contig 16
SEQ ID NO: 57: Contig 22
SEQ ID NO: 58: Contig 25
SEQ ID NO: 59: Contig 31
SEQ ID NO: 60: Contig 43
SEQ ID NO: 61: Forward primer of a positive control
SEQ ID NO: 62: Reverse primer of a positive control

1. A primer set for specifically detecting a nucleic acid derived from a lactic acid bacterium Enterococcus faecalis EF-2001 strain, the primer set comprising:
a pair of an oligonucleotide comprising the base sequence of SEQ ID NO: 1 and an oligonucleotide comprising the base sequence of SEQ ID NO: 2 or 3;
a pair of an oligonucleotide comprising the base sequence of SEQ ID NO: 4 and an oligonucleotide comprising the base sequence of SEQ ID NO: 5 or 6;
a pair of an oligonucleotide comprising the base sequence of SEQ ID NO: 7 and an oligonucleotide comprising the base sequence of SEQ ID NO: 8 or 9;
a pair of an oligonucleotide comprising the base sequence of SEQ ID NO: 10 and an oligonucleotide comprising the base sequence of SEQ ID NO: 11 or 12;
a pair of an oligonucleotide comprising the base sequence of SEQ ID NO: 13 and an oligonucleotide comprising the base sequence of SEQ ID NO: 14 or 15;
a pair of an oligonucleotide comprising the base sequence of SEQ ID NO: 16 and an oligonucleotide comprising the base sequence of SEQ ID NO: 17 or 18;
a pair of an oligonucleotide comprising the base sequence of SEQ ID NO: 19 and an oligonucleotide comprising the base sequence of SEQ ID NO: 20 or 21;
a pair of an oligonucleotide comprising the base sequence of SEQ ID NO: 22 and an oligonucleotide comprising the base sequence of SEQ ID NO: 23 or 24;
a pair of an oligonucleotide comprising the base sequence of SEQ ID NO: 25 and an oligonucleotide comprising the base sequence of SEQ ID NO: 26 or 27; or
a pair of an oligonucleotide comprising the base sequence of SEQ ID NO: 28 and an oligonucleotide comprising the base sequence of SEQ ID NO: 29 or 30.

2. A primer set for specifically detecting a nucleic acid derived from a lactic acid bacterium Enterococcus faecalis EF-2001 strain, the primer set selected from the group consisting of
(a) a primer set (1) for amplifying a part of the base sequence of SEQ ID NO: 51:
a forward primer
(SEQ ID NO: 1)
CGAAAAGGATGTAGTCAGCGG;
and
a reverse primer
(SEQ ID NO: 2)
CCGAAGGCGAAACAGAGGAT,
(b) a primer set (2) for amplifying a part of the base sequence of SEQ ID NO: 51:
a forward primer
(SEQ ID NO: 1)
CGAAAAGGATGTAGTCAGCGG;
and
a reverse primer
(SEQ ID NO: 3)
CCCAGACATAATCGCATGGC,
(c) a primer set (3) for amplifying a part of the base sequence of SEQ ID NO: 52:
a forward primer
(SEQ ID NO: 4)
GCGGCTGCACAATTTATTGC;
and
a reverse primer
(SEQ ID NO: 5)
AGAATACTTGGGCGGTCGTG,
(d) a primer set (4) for amplifying a part of the base sequence of SEQ ID NO: 52:
a forward primer
(SEQ ID NO: 4)
GCGGCTGCACAATTTATTGC;
and
a reverse primer
(SEQ ID NO: 6)
AATTCAGCTTCGCTAGATAAGGC,
(e) a primer set (5) for amplifying a part of the base sequence of SEQ ID NO: 53:
(SEQ ID NO: 7)
a forward primer CGCGTATGACTTGCAATCGA;
and
(SEQ ID NO: 8)
a reverse primer AGGATTGTTTGACGGTGCAA,
(f) a primer set (6) for amplifying a part of the base sequence of SEQ ID NO: 53:
(SEQ ID NO: 7)
a forward primer CGCGTATGACTTGCAATCGA;
and
(SEQ ID NO: 9)
a reverse primer ACATGAGATAGTTGGGGTAGACA,
(g) a primer set (7) for amplifying a part of the base sequence of SEQ ID NO: 54:
(SEQ ID NO: 10)
a forward primer CTTCAGAGAGCTGGGCGAAG;
and
(SEQ ID NO: 11)
a reverse primer TACTTTTTAGCTGCCCGCCC,
(h) a primer set (8) for amplifying a part of the base sequence of SEQ ID NO: 54:
(SEQ ID NO: 10)
a forward primer CTTCAGAGAGCTGGGCGAAG;
and
(SEQ ID NO: 12)
a reverse primer GGGTTGTAGCCCTACCCGAT,
(i) a primer set (9) for amplifying a part of the base sequence of SEQ ID NO: 55:
(SEQ ID NO: 13)
a forward primer CGTAACGTGACATTGCGGAC;
and
(SEQ ID NO: 14)
a reverse primer ATGCCAGTACGTCGCGTTAA,
(j) a primer set (10) for amplifying a part of the base sequence of SEQ ID NO: 55:
(SEQ ID NO: 13)
a forward primer CGTAACGTGACATTGCGGAC;
and
(SEQ ID NO: 15)
a reverse primer CGATTGTCAACTAATTGTGCCGA,
(k) a primer set (11) for amplifying a part of the base sequence of SEQ ID NO:
(SEQ ID NO: 16)
a forward primer CATGGCTTGCCGTTTCACAA;
and
(SEQ ID NO: 17)
a reverse primer ACCGCAACAACTACATACTACCA ,
(l) a primer set (12) for amplifying a part of the base sequence of SEQ ID NO: 56:
(SEQ ID NO: 16)
a forward primer CATGGCTTGCCGTTTCACAA;
and
(SEQ ID NO: 18)
a reverse primer ACCAAAAGGAACGCTACCAGT,
(m) a primer set (13) for amplifying a part of the base sequence of SEQ ID NO:
(SEQ ID NO: 19)
a forward primer TCAGCATAATCCCCAGACGT;
and
(SEQ ID NO: 20)
a reverse primer AATGAACGCCCTTCAGCAGA,
(n) a primer set (14) for amplifying a part of the base sequence of SEQ ID NO:
(SEQ ID NO: 19)
a forward primer TCAGCATAATCCCCAGACGT;
and
(SEQ ID NO: 21)
a reverse primer GGCTCCTCTACCTGAACAAACT,
(o) a primer set (15) for amplifying a part of the base sequence of SEQ ID NO:
(SEQ ID NO: 22)
a forward primer GCGTTCAAACTGTTCTGGTGT;
and
(SEQ ID NO: 23)
a reverse primer TACAAGGCTTGCGAGGTAGC,
(p) a primer set (16) for amplifying a part of the base sequence of SEQ ID NO:
(SEQ ID NO: 22)
a forward primer GCGTTCAAACTGTTCTGGTGT;
and
(SEQ ID NO: 24)
a reverse primer GCTGCAATGGAAAGCAAATCG,
(q) a primer set (17) for amplifying a part of the base sequence of SEQ ID NO:
(SEQ ID NO: 25)
a forward primer AGGCATATGGGTCATCTGCT;
and
(SEQ ID NO: 26)
a reverse primer GAGCATCACAGAGCCTCGAA,
(r) a primer set (18) for amplifying a part of the base sequence of SEQ ID NO:
(SEQ ID NO: 25)
a forward primer AGGCATATGGGTCATCTGCT;
and
(SEQ ID NO: 27)
a reverse primer AGAGATTTTTCAGTATTGCTGGGT,
(s) a primer set (19) for amplifying a part of the base sequence of SEQ ID NO:
a forward primer CGTTGGGTGTGCAGAAATGG (SEQ ID NO: 28); and
a reverse primer TGTACCGTCAACCTCGTTCG (SEQ ID NO: 29), or
(t) a primer set (20) for amplifying a part of the base sequence of SEQ ID NO: 60:
(SEQ ID NO: 28)
a forward primer CGTTGGGTGTGCAGAAATGG;
and
(SEQ ID NO: 30)
a reverse primer AACGGGTTGCGACTCTTTTT.

3. A kit for detecting a lactic acid bacterium Enterococcus faecalis EF-2001 strain, characterized by comprising the primer set of claim 1.

4. A method for specifically detecting a nucleic acid derived from a lactic acid bacterium Enterococcus faecalis EF-2001 strain, the method comprising:
the step of amplifying a nucleic acid fragment using the primer set of claim 1; and
the step of detecting a nucleic acid fragment obtained in the amplification step.

5. A lactic acid bacterium composition comprising a nucleic acid fragment,
wherein the nucleic acid fragment is not amplified by one or more primer sets selected from the group consisting of:
a pair of an oligonucleotide comprising the base sequence of SEQ ID NO: 1 and an oligonucleotide comprising the base sequence of SEQ ID NO: 2;
a pair of an oligonucleotide comprising the base sequence of SEQ ID NO: 4 and an oligonucleotide comprising the base sequence of SEQ ID NO: 5;
a pair of an oligonucleotide comprising the base sequence of SEQ ID NO: 7 and an oligonucleotide comprising the base sequence of SEQ ID NO: 8;
a pair of an oligonucleotide comprising the base sequence of SEQ ID NO: 10 and an oligonucleotide comprising the base sequence of SEQ ID NO: 11;
a pair of an oligonucleotide comprising the base sequence of SEQ ID NO: 13 and an oligonucleotide comprising the base sequence of SEQ ID NO: 14;
a pair of an oligonucleotide comprising the base sequence of SEQ ID NO: 16 and an oligonucleotide comprising the base sequence of SEQ ID NO: 17;
a pair of an oligonucleotide comprising the base sequence of SEQ ID NO: 19 and an oligonucleotide comprising the base sequence of SEQ ID NO: 20;
a pair of an oligonucleotide comprising the base sequence of SEQ ID NO: 22 and an oligonucleotide comprising the base sequence of SEQ ID NO: 23;
a pair of an oligonucleotide comprising the base sequence of SEQ ID NO: 25 and an oligonucleotide comprising the base sequence of SEQ ID NO: 26; and/or
a pair of an oligonucleotide comprising the base sequence of SEQ ID NO: 28 and an oligonucleotide comprising the base sequence of SEQ ID NO: 29, and
wherein the nucleic acid fragment is amplified by one or more primer sets selected from the group consisting of:
a pair of an oligonucleotide comprising the base sequence of SEQ ID NO: 1 and an oligonucleotide comprising the base sequence of SEQ ID NO: 3;
a pair of an oligonucleotide comprising the base sequence of SEQ ID NO: 4 and an oligonucleotide comprising the base sequence of SEQ ID NO: 6;
a pair of an oligonucleotide comprising the base sequence of SEQ ID NO: 7 and an oligonucleotide comprising the base sequence of SEQ ID NO: 9;
a pair of an oligonucleotide comprising the base sequence of SEQ ID NO: 10 and an oligonucleotide comprising the base sequence of SEQ ID NO: 12;
a pair of an oligonucleotide comprising the base sequence of SEQ ID NO: 13 and an oligonucleotide comprising the base sequence of SEQ ID NO: 15;
a pair of an oligonucleotide comprising the base sequence of SEQ ID NO: 16 and an oligonucleotide comprising the base sequence of SEQ ID NO: 18;
a pair of an oligonucleotide comprising the base sequence of SEQ ID NO: 19 and an oligonucleotide comprising the base sequence of SEQ ID NO: 21;
a pair of an oligonucleotide comprising the base sequence of SEQ ID NO: 22 and an oligonucleotide comprising the base sequence of SEQ ID NO: 24;
a pair of an oligonucleotide comprising the base sequence of SEQ ID NO: 25 and an oligonucleotide comprising the base sequence of SEQ ID NO: 27; and/or
a pair of an oligonucleotide comprising the base sequence of SEQ ID NO: 28 and an oligonucleotide comprising the base sequence of SEQ ID NO: 30.

6. The composition of claim 5, comprising a nucleic acid fragment which is not amplified by a primer set of an oligonucleotide comprising the base sequence of SEQ ID NO: 16 and an oligonucleotide comprising the base sequence of SEQ ID NO: 17 and is amplified by a primer set of an oligonucleotide comprising the base sequence of SEQ ID NO: 16 and an oligonucleotide comprising the base sequence of SEQ ID NO: 18.

7. The composition of claim 5, comprising a heat-treated lactic acid bacterium product resulting from subjecting a lactic acid bacterium Enterococcus faecalis EF-2001 strain to heat treatment at at least about 70° C.

8. The composition of claim 5, comprising a heat-treated lactic acid bacterium product resulting from subjecting a lactic acid bacterium Enterococcus faecalis EF-2001 strain to heat treatment at at least about 90° C.

9. A method for manufacturing a lactic acid bacterium composition, the method comprising:
the step of heating a lactic acid bacterium Enterococcus faecalis EF-2001 strain to obtain a plurality of lots of a heat-treated lactic acid bacterium product;
the step of extracting a nucleic acid fragment in one of the lots of the heat-treated lactic acid bacterium product;
the step of amplifying the nucleic acid fragment using at least one of the primer sets of claim 1;
the step of detecting a nucleic acid fragment obtained in the amplification step; and
the step of selecting a lot of the heat-treated lactic acid bacterium product in which the nucleic acid fragment has been detected.

10. The method of claim 9, wherein the heating is heating at at least about 70° C.

11. The method of claim 9, wherein the heating is heating at at least about 90° C.

12. The method of claim 9, wherein the nucleic acid fragment is amplified by one or more primer sets selected from the group consisting of:
a pair of an oligonucleotide comprising the base sequence of SEQ ID NO: 1 and an oligonucleotide comprising the base sequence of SEQ ID NO: 3;
a pair of an oligonucleotide comprising the base sequence of SEQ ID NO: 4 and an oligonucleotide comprising the base sequence of SEQ ID NO: 6;
a pair of an oligonucleotide comprising the base sequence of SEQ ID NO: 7 and an oligonucleotide comprising the base sequence of SEQ ID NO: 9;
a pair of an oligonucleotide comprising the base sequence of SEQ ID NO: 10 and an oligonucleotide comprising the base sequence of SEQ ID NO: 12;
a pair of an oligonucleotide comprising the base sequence of SEQ ID NO: 13 and an oligonucleotide comprising the base sequence of SEQ ID NO: 15;
a pair of an oligonucleotide comprising the base sequence of SEQ ID NO: 16 and an oligonucleotide comprising the base sequence of SEQ ID NO: 18;
a pair of an oligonucleotide comprising the base sequence of SEQ ID NO: 19 and an oligonucleotide comprising the base sequence of SEQ ID NO: 21;
a pair of an oligonucleotide comprising the base sequence of SEQ ID NO: 22 and an oligonucleotide comprising the base sequence of SEQ ID NO: 24;
a pair of an oligonucleotide comprising the base sequence of SEQ ID NO: 25 and an oligonucleotide comprising the base sequence of SEQ ID NO: 27; and/or
a pair of an oligonucleotide comprising the base sequence of SEQ ID NO: 28 and an oligonucleotide comprising the base sequence of SEQ ID NO: 30, and
wherein the nucleic acid fragment is not amplified by one or more primer sets selected from the group consisting of:
a pair of an oligonucleotide comprising the base sequence of SEQ ID NO: 1 and an oligonucleotide comprising the base sequence of SEQ ID NO: 2;
a pair of an oligonucleotide comprising the base sequence of SEQ ID NO: 4 and an oligonucleotide comprising the base sequence of SEQ ID NO: 5;
a pair of an oligonucleotide comprising the base sequence of SEQ ID NO: 7 and an oligonucleotide comprising the base sequence of SEQ ID NO: 8;
a pair of an oligonucleotide comprising the base sequence of SEQ ID NO: 10 and an oligonucleotide comprising the base sequence of SEQ ID NO: 11;
a pair of an oligonucleotide comprising the base sequence of SEQ ID NO: 13 and an oligonucleotide comprising the base sequence of SEQ ID NO: 14;
a pair of an oligonucleotide comprising the base sequence of SEQ ID NO: 16 and an oligonucleotide comprising the base sequence of SEQ ID NO: 17;
a pair of an oligonucleotide comprising the base sequence of SEQ ID NO: 19 and an oligonucleotide comprising the base sequence of SEQ ID NO: 20;
a pair of an oligonucleotide comprising the base sequence of SEQ ID NO: 22 and an oligonucleotide comprising the base sequence of SEQ ID NO: 23;
a pair of an oligonucleotide comprising the base sequence of SEQ ID NO: 25 and an oligonucleotide comprising the base sequence of SEQ ID NO: 26; and/or
a pair of an oligonucleotide comprising the base sequence of SEQ ID NO: 28 and an oligonucleotide comprising the base sequence of SEQ ID NO: 29.

13. A polynucleotide characterized by being amplified by a primer set for detecting a lactic acid bacterium Enterococcus faecalis EF-2001 strain, wherein the polynucleotide is:
the polynucleotide set forth in SEQ ID NO: 31 which is amplified by a primer set consisting of a pair of an oligonucleotide comprising the base sequence of SEQ ID NO: 1 and an oligonucleotide comprising the base sequence of SEQ ID NO: 2;
the polynucleotide set forth in SEQ ID NO: 32 which is amplified by a primer set consisting of a pair of an oligonucleotide comprising the base sequence of SEQ ID NO: 1 and an oligonucleotide comprising the base sequence of SEQ ID NO: 3;
the polynucleotide set forth in SEQ ID NO: 33 which is amplified by a primer set consisting of a pair of an oligonucleotide comprising the base sequence of SEQ ID NO: 4 and an oligonucleotide comprising the base sequence of SEQ ID NO: 5;
the polynucleotide set forth in SEQ ID NO: 34 which is amplified by a primer set consisting of a pair of an oligonucleotide comprising the base sequence of SEQ ID NO: 4 and an oligonucleotide comprising the base sequence of SEQ ID NO: 6;
the polynucleotide set forth in SEQ ID NO: 35 which is amplified by a primer set consisting of a pair of an oligonucleotide comprising the base sequence of SEQ ID NO: 7 and an oligonucleotide comprising the base sequence of SEQ ID NO: 8;
the polynucleotide set forth in SEQ ID NO: 36 which is amplified by a primer set consisting of a pair of an oligonucleotide comprising the base sequence of SEQ ID NO: 7 and an oligonucleotide comprising the base sequence of SEQ ID NO: 9;
the polynucleotide set forth in SEQ ID NO: 37 which is amplified by a primer set consisting of a pair of an oligonucleotide comprising the base sequence of SEQ ID NO: 10 and an oligonucleotide comprising the base sequence of SEQ ID NO: 11;
the polynucleotide set forth in SEQ ID NO: 38 which is amplified by a primer set consisting of a pair of an oligonucleotide comprising the base sequence of SEQ ID NO: 10 and an oligonucleotide comprising the base sequence of SEQ ID NO: 12;
the polynucleotide set forth in SEQ ID NO: 39 which is amplified by a primer set consisting of a pair of an oligonucleotide comprising the base sequence of SEQ ID NO: 13 and an oligonucleotide comprising the base sequence of SEQ ID NO: 14;
the polynucleotide set forth in SEQ ID NO: 40 which is amplified by a primer set consisting of a pair of an oligonucleotide comprising the base sequence of SEQ ID NO: 13 and an oligonucleotide comprising the base sequence of SEQ ID NO: 15;
the polynucleotide set forth in SEQ ID NO: 41 which is amplified by a primer set consisting of a pair of an oligonucleotide comprising the base sequence of SEQ ID NO: 16 and an oligonucleotide comprising the base sequence of SEQ ID NO: 17;
the polynucleotide set forth in SEQ ID NO: 42 which is amplified by a primer set consisting of a pair of an oligonucleotide comprising the base sequence of SEQ ID NO: 16 and an oligonucleotide comprising the base sequence of SEQ ID NO: 18;
the polynucleotide set forth in SEQ ID NO: 43 which is amplified by a primer set consisting of a pair of an oligonucleotide comprising the base sequence of SEQ ID NO: 19 and an oligonucleotide comprising the base sequence of SEQ ID NO: 20;
the polynucleotide set forth in SEQ ID NO: 44 which is amplified by a primer set consisting of a pair of an oligonucleotide comprising the base sequence of SEQ ID NO: 19 and an oligonucleotide comprising the base sequence of SEQ ID NO: 21;
the polynucleotide set forth in SEQ ID NO: 45 which is amplified by a primer set consisting of a pair of an oligonucleotide comprising the base sequence of SEQ ID NO: 22 and an oligonucleotide comprising the base sequence of SEQ ID NO: 23;
the polynucleotide set forth in SEQ ID NO: 46 which is amplified by a primer set consisting of a pair of an oligonucleotide comprising the base sequence of SEQ ID NO: 22 and an oligonucleotide comprising the base sequence of SEQ ID NO: 24;
the polynucleotide set forth in SEQ ID NO: 47 which is amplified by a primer set consisting of a pair of an oligonucleotide comprising the base sequence of SEQ ID NO: 25 and an oligonucleotide comprising the base sequence of SEQ ID NO: 26;
the polynucleotide set forth in SEQ ID NO: 48 which is amplified by a primer set consisting of a pair of an oligonucleotide comprising the base sequence of SEQ ID NO: 25 and an oligonucleotide comprising the base sequence of SEQ ID NO: 27;
the polynucleotide set forth in SEQ ID NO: 49 which is amplified by a primer set consisting of a pair of an oligonucleotide comprising the base sequence of SEQ ID NO: 28 and an oligonucleotide comprising the base sequence of SEQ ID NO: 29; or
the polynucleotide set forth in SEQ ID NO: 50 which is amplified by a primer set consisting of a pair of an oligonucleotide comprising the base sequence of SEQ ID NO: 28 and an oligonucleotide comprising the base sequence of SEQ ID NO: 30.

14. A kit for detecting a lactic acid bacterium Enterococcus faecalis EF-2001 strain, characterized by comprising the primer set of claim 2.

15. A method for specifically detecting a nucleic acid derived from a lactic acid bacterium Enterococcus faecalis EF-2001 strain, the method comprising:
the step of amplifying a nucleic acid fragment using the primer set of claim 2; and
the step of detecting a nucleic acid fragment obtained in the amplification step.

16. A method for manufacturing a lactic acid bacterium composition, the method comprising:
the step of heating a lactic acid bacterium Enterococcus faecalis EF-2001 strain to obtain a plurality of lots of a heat-treated lactic acid bacterium product;
the step of extracting a nucleic acid fragment in one of the lots of the heat-treated lactic acid bacterium product;
the step of amplifying the nucleic acid fragment using at least one of the primer sets of claim 2;
the step of detecting a nucleic acid fragment obtained in the amplification step; and
the step of selecting a lot of the heat-treated lactic acid bacterium product in which the nucleic acid fragment has been detected.

17. The method of claim 16, wherein the heating is heating at at least about 70° C.

18. The method of claim 16, wherein the heating is heating at at least about 90° C.

19. The method of claim 16, wherein the nucleic acid fragment is amplified by one or more primer sets selected from the group consisting of:
a pair of an oligonucleotide comprising the base sequence of SEQ ID NO: 1 and an oligonucleotide comprising the base sequence of SEQ ID NO: 3;
a pair of an oligonucleotide comprising the base sequence of SEQ ID NO: 4 and an oligonucleotide comprising the base sequence of SEQ ID NO: 6;
a pair of an oligonucleotide comprising the base sequence of SEQ ID NO: 7 and an oligonucleotide comprising the base sequence of SEQ ID NO: 9;
a pair of an oligonucleotide comprising the base sequence of SEQ ID NO: 10 and an oligonucleotide comprising the base sequence of SEQ ID NO: 12;
a pair of an oligonucleotide comprising the base sequence of SEQ ID NO: 13 and an oligonucleotide comprising the base sequence of SEQ ID NO: 15;
a pair of an oligonucleotide comprising the base sequence of SEQ ID NO: 16 and an oligonucleotide comprising the base sequence of SEQ ID NO: 18;
a pair of an oligonucleotide comprising the base sequence of SEQ ID NO: 19 and an oligonucleotide comprising the base sequence of SEQ ID NO: 21;
a pair of an oligonucleotide comprising the base sequence of SEQ ID NO: 22 and an oligonucleotide comprising the base sequence of SEQ ID NO: 24;
a pair of an oligonucleotide comprising the base sequence of SEQ ID NO: 25 and an oligonucleotide comprising the base sequence of SEQ ID NO: 27; and/or
a pair of an oligonucleotide comprising the base sequence of SEQ ID NO: 28 and an oligonucleotide comprising the base sequence of SEQ ID NO: 30, and
wherein the nucleic acid fragment is not amplified by one or more primer sets selected from the group consisting of:
a pair of an oligonucleotide comprising the base sequence of SEQ ID NO: 1 and an oligonucleotide comprising the base sequence of SEQ ID NO: 2;
a pair of an oligonucleotide comprising the base sequence of SEQ ID NO: 4 and an oligonucleotide comprising the base sequence of SEQ ID NO: 5;
a pair of an oligonucleotide comprising the base sequence of SEQ ID NO: 7 and an oligonucleotide comprising the base sequence of SEQ ID NO: 8;
a pair of an oligonucleotide comprising the base sequence of SEQ ID NO: 10 and an oligonucleotide comprising the base sequence of SEQ ID NO: 11;
a pair of an oligonucleotide comprising the base sequence of SEQ ID NO: 13 and an oligonucleotide comprising the base sequence of SEQ ID NO: 14;
a pair of an oligonucleotide comprising the base sequence of SEQ ID NO: 16 and an oligonucleotide comprising the base sequence of SEQ ID NO: 17;
a pair of an oligonucleotide comprising the base sequence of SEQ ID NO: 19 and an oligonucleotide comprising the base sequence of SEQ ID NO: 20;
a pair of an oligonucleotide comprising the base sequence of SEQ ID NO: 22 and an oligonucleotide comprising the base sequence of SEQ ID NO: 23;
a pair of an oligonucleotide comprising the base sequence of SEQ ID NO: 25 and an oligonucleotide comprising the base sequence of SEQ ID NO: 26; and/or
a pair of an oligonucleotide comprising the base sequence of SEQ ID NO: 28 and an oligonucleotide comprising the base sequence of SEQ ID NO: 29.


SEQUENCE LISTING (62 sequences)

SEQ ID NO: 1 - DNA, 21 bp, Artificial Sequence
  Contig_01 Forward Primer
cgaaaaggatgtagtcagcgg

SEQ ID NO: 2 - DNA, 20 bp, Artificial Sequence
  Contig_01 Reverse Primer for Long
ccgaaggcgaaacagaggat

SEQ ID NO: 3 - DNA, 20 bp, Artificial Sequence
  Contig_01 Reverse Primer for Short
cccagacataatcgcatggc

SEQ ID NO: 4 - DNA, 20 bp, Artificial Sequence
  Contig_04 Forward Primer
gcggctgcacaatttattgc

SEQ ID NO: 5 - DNA, 20 bp, Artificial Sequence
  Contig_04 Reverse Primer for Long
agaatacttgggcggtcgtg

SEQ ID NO: 6 - DNA, 23 bp, Artificial Sequence
  Contig_04 Reverse Primer for Short
aattcagcttcgctagataaggc

SEQ ID NO: 7 - DNA, 20 bp, Artificial Sequence
  Contig_07 Forward Primer
cgcgtatgacttgcaatcga

SEQ ID NO: 8 - DNA, 20 bp, Artificial Sequence
  Contig_07 Reverse Primer for Long
aggattgtttgacggtgcaa

SEQ ID NO: 9 - DNA, 23 bp, Artificial Sequence
  Contig_07 Reverse Primer for Short
acatgagatagttggggtagaca

SEQ ID NO: 10 - DNA, 20 bp, Artificial Sequence
  Contig_11 Forward Primer
cttcagagagctgggcgaag

SEQ ID NO: 11 - DNA, 20 bp, Artificial Sequence
  Contig_11 Reverse Primer for Long
tactttttagctgcccgccc

SEQ ID NO: 12 - DNA, 20 bp, Artificial Sequence
  Contig_11 Reverse Primer for Short
gggttgtagccctacccgat

SEQ ID NO: 13 - DNA, 20 bp, Artificial Sequence
  Contig_13 Forward Primer
cgtaacgtgacattgcggac

SEQ ID NO: 14 - DNA, 20 bp, Artificial Sequence
  Contig_13 Reverse Primer for Long
atgccagtacgtcgcgttaa

SEQ ID NO: 15 - DNA, 23 bp, Artificial Sequence
  Contig_13 Reverse Primer for Short
cgattgtcaactaattgtgccga

SEQ ID NO: 16 - DNA, 20 bp, Artificial Sequence
  Contig_16 Forward Primer
catggcttgccgtttcacaa

SEQ ID NO: 17 - DNA, 23 bp, Artificial Sequence
  Contig_16 Reverse Primer for Long
accgcaacaactacatactacca

SEQ ID NO: 18 - DNA, 21 bp, Artificial Sequence
  Contig_16 Reverse Primer for Short
accaaaaggaacgctaccagt

SEQ ID NO: 19 - DNA, 20 bp, Artificial Sequence
  Contig_22 Forward Primer
tcagcataatccccagacgt

SEQ ID NO: 20 - DNA, 20 bp, Artificial Sequence
  Contig_22 Reverse Primer for Long
aatgaacgcccttcagcaga

SEQ ID NO: 21 - DNA, 22 bp, Artificial Sequence
  Contig_22 Reverse Primer for Short
ggctcctctacctgaacaaact

SEQ ID NO: 22 - DNA, 21 bp, Artificial Sequence
  Contig_25 Forward Primer
gcgttcaaactgttctggtgt

SEQ ID NO: 23 - DNA, 20 bp, Artificial Sequence
  Contig_25 Reverse Primer for Long
tacaaggcttgcgaggtagc

SEQ ID NO: 24 - DNA, 21 bp, Artificial Sequence
  Contig_25 Reverse Primer for Short
gctgcaatggaaagcaaatcg

SEQ ID NO: 25 - DNA, 20 bp, Artificial Sequence
  Contig_31 Forward Primer
aggcatatgggtcatctgct

SEQ ID NO: 26 - DNA, 20 bp, Artificial Sequence
  Contig_31 Reverse Primer for Long
gagcatcacagagcctcgaa

SEQ ID NO: 27 - DNA, 24 bp, Artificial Sequence
  Contig_31 Reverse Primer for Short
agagatttttcagtattgctgggt

SEQ ID NO: 28 - DNA, 20 bp, Artificial Sequence
  Contig_43 Forward Primer
cgttgggtgtgcagaaatgg

SEQ ID NO: 29 - DNA, 20 bp, Artificial Sequence
  Contig_43 Reverse Primer for Long
tgtaccgtcaacctcgttcg

SEQ ID NO: 30 - DNA, 20 bp, Artificial Sequence
  Contig_43 Reverse Primer for Short
aacgggttgcgactcttttt

SEQ ID NO: 31 - DNA, 581 bp, Enterococcus faecalis
cgaaaaggatgtagtcagcggattattcaaccgcataatactcatcctttctttttgctaactttttttagatattcttgttttaatttttttgattttttaccacgctcatctaataattcagtctgtttcttagtgtattcttcaatagattcactaatacctactgataaatcgtaaaaggcgtttagccatgcgattatgtctgggatgtctttatacaaaattgcaaacgtaccttcaccaagtacggaatcatacccttttgtcaacacagaaatatataattcatcactcatttcctcagaattttcatctaagtctttcagttcttgtacgactttatcgtacgtttcttcatattcttttatatgctctaatgaacaatcaaagaaaaattcgtgcccagcaattttcactggaaaaccagtacgctctactttaatctctaatgctttcatttaattacctccataaataaggacagccaaatagctgcccttattttcgtatttatgcttgattattaagtgttaaggtatgttgtgctgttttcttaccatcctctgtttcgccttcgg

SEQ ID NO: 32 - DNA, 210 bp, Enterococcus faecalis
cgaaaaggatgtagtcagcggattattcaaccgcataatactcatcctttctttttgctaactttttttagatattcttgttttaatttttttgattttttaccacgctcatctaataattcagtctgtttcttagtgtattcttcaatagattcactaatacctactgataaatcgtaaaaggcgtttagccatgcgattatgtctggg

SEQ ID NO: 33 - DNA, 594 bp, Enterococcus faecalis
gcggctgcacaatttattgcagctgactatgatgctgatatagctgaagaattcaccaatacttagagagccaatatattagactctcttttttaattcttcaataagaacacggtttgtttctaacgccttatctagcgaagctgaattaatttctaataaagatttcaaaaattcgacttcatttttcagctctgaaatctttattttttcgtaagtttcttcagtcacttccattttttcctacctcctgatcttctattggcgtatattcttgtttgacgtaaaacggaagatctgttaattcttcaaaatcaaatttttcacctataacgcttcttaagtcgtcatcatcttgaaaatagatagaatcaactttctccccttcaattagatcaaaagcatcttgtaattcgattggtaaataaattggacgtttacccatttaaatttctcctttcaaaagaaaaggggcgtttccgccccctattcagtttttagttaaacatgtcatcatcgttatcttcttcttcgtcatcccaatccaaatcaccgaagtcagattctgcatttgcacgaccgcccaagtattct

SEQ ID NO: 34 - DNA, 150 bp, Enterococcus faecalis
gcggctgcacaatttattgcagctgactatgatgctgatatagctgaagaattcaccaatacttagagagccaatatattagactctcttttttaattcttcaataagaacacggtttgtttctaacgccttatctagcgaagctgaatt

SEQ ID NO: 35 - DNA, 510 bp, Enterococcus faecalis
cgcgtatgacttgcaatcgaagcatctataaatttagaatacaaaatttgaataatcaattctttctgagattcaaaagtatttaaaatcatttcggataacgttattagactctcttcactcgaatcttgattttctataacggttaaaaccttttttatgtctaccccaactatctcatgtacatcagataacccatacttacagcatctactaataaaagtaatcaactctctatcatcaatctctatttctgtaggatgtgctactgatgcattgtttctaatttctctacattagtttagataaaaatttccttcctcattaataatattgagctgataacatagttctattaattctgaatctgttattttatccaaaaaattatcatctttttttgccccagaaatattcttaatcatttccaatccaaaagaactaactttatttctcaattctttgatcacactattccatatgtatactattgcaccgtcaaacaatcct

SEQ ID NO: 36 - DNA, 183 bp, Enterococcus faecalis
cgcgtatgacttgcaatcgaagcatctataaatttagaatacaaaatttgaataatcaattctttctgagattcaaaagtatttaaaatcatttcggataacgttattagactctcttcactcgaatcttgattttctataacggttaaaaccttttttatgtctaccccaactatctcatgt

SEQ ID NO: 37 - DNA, 513 bp, Enterococcus faecalis
cttcagagagctgggcgaagggatcagagaaaaaagcgaaagaacaattaaataacgccttctcatattatcctaaaaattgggctgacttattggcaagaaatgataaaaaactattttcgggggttaataaaagaggctttttctttaacggtttgcttgataaaaggggacgatataaaacaaatattgaggggtttaaaacagattttgttaccatccatttatcgggtagggctacaaccccttttcatgagataggacatctgattgagtgttttaaccctgatgtcgtaagggtagaaaaagaatttatcgaagctcgaactagaggggagataccgactcgattgtcagaaatattcccagggagcggttatagcaatcgtgaagtaactaaaaaagataattttatcagtccatatataggtaaggtatatcctaatgccacagaagttttaagtgtcggtttggaaagtttatttgagccacagggcgggcagctaaaaagta

SEQ ID NO: 38 - DNA, 246 bp, Enterococcus faecalis
cttcagagagctgggcgaagggatcagagaaaaaagcgaaagaacaattaaataacgccttctcatattatcctaaaaattgggctgacttattggcaagaaatgataaaaaactattttcgggggttaataaaagaggctttttctttaacggtttgcttgataaaaggggacgatataaaacaaatattgaggggtttaaaacagattttgttaccatccatttatcgggtagggctacaaccc

SEQ ID NO: 39 - DNA, 600 bp, Enterococcus faecalis
cgtaacgtgacattgcggacttagctatccctactttttctgctaattcagtggatgtcatttttttctgtttgcgcaaatctgtaattaatgtcatgatctcatcgtttgttctcaaattattcacctcctaagagaataatactaataaagttcccaatagtcaacaaaaaatgttctaattcggcacaattagttgacaatcgggaattgtcgatatatcattaaagcacattcatggatgaataaaaatcaaggagggaattattaatatggaatttgaactcaatcgtttaaaggctgaaaggattgctaagggttattcgcaagaagaattagcaaaaaaattaggttggaccagaagtatgtatacaaaacgtgaaaatggtgcagtttcattgggagttgatgaactagcaaaaatagcaacagcattagaaatgccagaatctagaatatctatttttttttaatcgttgagttcccgaatgtcaacaaaggaggagggaagtgtgatatgaatcaaaagtcgtcagaaaaagtagatatcgatttattggcacaagcttttgctgatgttttaacgcgacgtactggcat

SEQ ID NO: 40 - DNA, 206 bp, Enterococcus faecalis
cgtaacgtgacattgcggacttagctatccctactttttctgctaattcagtggatgtcatttttttctgtttgcgcaaatctgtaattaatgtcatgatctcatcgtttgttctcaaattattcacctcctaagagaataatactaataaagttcccaatagtcaacaaaaaatgttctaattcggcacaattagttgacaatcg

SEQ ID NO: 41 - DNA, 509 bp, Enterococcus faecalis
catggcttgccgtttcacaaccatttttcaatactgatactgcttatgggcgttcctttgttaatcagtcaatgagttttgctgaattagaagcacaaatggcatctgaacgtatcaaagcagtatttgaaaacaaaattagaaagggagaagtggtaactggtagcgttccttttggttataaaatctgtgataagaagttaatacctaacgaaaatgcacctatagcaaaagacatttttaaacattattctattcacaacagtatacgcctaactgttgaatatctattcaatgaatatgatattacaagaagttctcgaacaatcaagcacatgttaaggaatagaaaatatataggtgaagtttctggtaacaaaaattattgtcctcccatagtagataaggaaacctttgagaaggttcaaaatctattagataaaaatatttcatctatagcaaaacgtacttatatcttttcaggactggtagtatgtagttgttgcggt

SEQ ID NO: 42 - DNA, 179 bp, Enterococcus faecalis
catggcttgccgtttcacaaccatttttcaatactgatactgcttatgggcgttcctttgttaatcagtcaatgagttttgctgaattagaagcacaaatggcatctgaacgtatcaaagcagtatttgaaaacaaaattagaaagggagaagtggtaactggtagcgttccttttggt

SEQ ID NO: 43 - DNA, 532 bp, Enterococcus faecalis
tcagcataatccccagacgttacaacttcaaaatcaaaattacgactgttacctttagtgttaaatccaactactttatatttataacctttcccgaaaaatttaccactgtcatcttttaagtcagtttgttcaggtagaggagcctgtattttagcataatagtcttctcctacgtaagtagattgatagtaatcccaacctttccatccaataaatataagtaacgcaacaaaaattaagcttatgatttttttcatgtgaaaacctcctaaagtatttaatatatttaatttacatgaatcaaactaataagttaatcgaaataactaacatttttgtaataaatttattatccgattagaaagtgagtgaagaagatgattccaaaaatagaagtatggatgcatgatatgtccgttggctatcctgtgtggtttgaagtagattcaattgattatctagaaaattcgtttgttatagtagatgaatttggtagtccgcatgagttctctgctgaagggcgttcatt

SEQ ID NO: 44 - DNA, 147 bp, Enterococcus faecalis
tcagcataatccccagacgttacaacttcaaaatcaaaattacgactgttacctttagtgttaaatccaactactttatatttataacctttcccgaaaaatttaccactgtcatcttttaagtcagtttgttcaggtagaggagcc

SEQ ID NO: 45 - DNA, 548 bp, Enterococcus faecalis
gcgttcaaactgttctggtgttagttctaagtacttgtctatagttgggttaaacaaaatatacctcctttaaaaagactattaataaatctaaaatcttctatcttttgatagtctccagtaatgtaaacccaaagctgttagtttacacgatttgctttccattgcagcaaaatacatatgattagtatcaacaggttcgattaaaccaacactctcatataattgcagagttttaaatttagaaacattattttcatctgcatagggtttaattacttgatgtacttgagatggatcatttgtaaattcaaaagatggatctaagcgaaactcatcactaggatttgggaagaattgtgacaattgtcgcaaagttttctttgatactcttggttcaatagaacgtataggtaaaaaacttgaaatattagttttaaatactggacgttgttcccaagcacccaaagactgatctatgaatgagtataagctggctggtgttatttttccagttatatctgaggcgctacctcgcaagccttgta

SEQ ID NO: 46 - DNA, 171 bp, Enterococcus faecalis
gcgttcaaactgttctggtgttagttctaagtacttgtctatagttgggttaaacaaaatatacctcctttaaaaagactattaataaatctaaaatcttctatcttttgatagtctccagtaatgtaaacccaaagctgttagtttacacgatttgctttccattgcagc

SEQ ID NO: 47 - DNA, 575 bp, Enterococcus faecalis
aggcatatgggtcatctgctagtcgtatcaaagaatcaatataattagcttgaatactaccgttctcaatagaagataagttactataactaatacccagcaatactgaaaaatctcttatagttagttcgtatttttctctaatttcttttacttttttagcagataacaaattgtgtctttctctgtaaatattatagtctttttctaaattttgatctgggttagaaaatggttcaaataattcatcatctgatattcttttataatattcatgtttgactttaaatgtatcgtttttaattgtaacgtcttcttctaaaaaaattacttcgtatctttcattttgattagtttcatatgaaaacttttctataattttagacatactaaatcacctctcccttatagtctaatggaaagcaatcaatttcttgttctctaggatgaaaagacatatatgctgcaacaaaattattctcttgtgcaatcaattccaacttcacatacacttttatttcatcccaaactaagccgaattctgtcacagttctattcgaggctctgtgatgctc

SEQ ID NO: 48 - DNA, 118 bp, Enterococcus faecalis
aggcatatgggtcatctgctagtcgtatcaaagaatcaatataattagcttgaatactaccgttctcaatagaagataagttactataactaatacccagcaatactgaaaaatctct

SEQ ID NO: 49 - DNA, 593 bp, Enterococcus faecalis
cgttgggtgtgcagaaatgggacttagcatatgagaattataaaaatggtatgaagtataaagatatagcagataaatacgatgtatctattaacaccgtgaagtcttggaaatctcgcaaatggaacgcgactcctaaagaggttgcaaccaaaaagaaaaaggttgcacacaaaaaagagtcgcaacccgttatagagaatgatgctttaacagagcaacagaaaatgttctgtttattttatttacagcattttaacgccactaaggcatatcaacaagcatacggatgtggctataattcagctagagccaatagcattaggttgctagcaaaagatcgcataaaaaaagagttgcaccgtttaaaagcagagttgcaacaagatgtgtttgtggatattaaagacttgatacaagagtatgttaagcaagcatttgccgatattaccgattttacagaatttggttatgatgaatatccgtttaaagatattaatggtgaagaggttatagacgaagaaacaggcgaagtaaaaacatacaaagtttcaaatgtctctcttaaagattcgaacgaggttgacggtaca

SEQ ID NO: 50 - DNA, 194 bp, Enterococcus faecalis
cgttgggtgtgcagaaatgggacttagcatatgagaattataaaaatggtatgaagtataaagatatagcagataaatacgatgtatctattaacaccgtgaagtcttggaaatctcgcaaatggaacgcgactcctaaagaggttgcaaccaaaaagaaaaaggttgcacacaaaaaagagtcgcaacccgtt

SEQ ID NO: 51 - DNA, 4408 bp, Enterococcus faecalis
ttgtaggattgtaggttttcgcaaagttgctattatttacaatttggctacccaaattagcagcgcccatcatatttgtgtttaatgctaattctggactagtaaagttaaaatttgaaaaagcattgtcaaatgagagttccttagaaatagaatctgccatattagaagtaagatgtttaacattatcaaatcctgctattattcccttttctaaaccaaacattagtgcagaaccattatcaattaaaacttttttatcatatggaattggaccttttaaactagcgatagtatctgcgataccactaacaaagcctttaactttttcaaatccccattttattccttttagtaatccacccattaaagatgtacctgcatcccatagttcatctccaacaattgcagaacctaaactatttactaatctttttactgcctctctagcctcttctttattattatcaataccctctgctaaaccattaattaatcgtatagctgctttccacattctatctgagaaattaataatattctcaactaatttatcaactaaatctgcagcagcgtttactaatctgtataaattatttccaattccttctatacatttcactactaaatccatacctgcatcgacgattcgtgatagattattagaaataccttgtagtacgctaacgattaacgtcactccggcatcaatgatatcaggcattctagaagataatccatttaagaaattaatcatcaaattaacagccgcatcgataatatctcccattctattaccgattgcattaacaaaattaacgataatactaattgcttcattagtgactttgccaatattatttgcaattccacgaagaaaattaatcaacaaattgaaaccagcttgcaaaatatcaggcaaatgtttattcaactctgaaagccacgtgataattaatgtagccatgttttgcactattttaggtaattgttctgttataccttgaagcaatgcattgattaaactagctccagctcctaacaactttggtagtgcttcagtaagtgaacctaaaaaagtagtaataatcatcgttgctgattctatgatagtcggcataactaataagataccattagtcagtgcattgattatttgtactgcagattgagcaatgataggcaacagagtgactattccattagtaaacgccattatcagttccccagcagatactacaatgcttggcaatccttgtgcgataccagaaataaaaccagacactacttgtaaagctccagtaataatacctggtaaagcattggcaatcgcacctaaaattccttgtagcgctgtaccaaaatttgtacctaattgtggcccataagtcttgattccttcagctaatcctttaaaagaattcataattgtatcaatacctttatttacatctccgccacccagagcttttgctagtaattcaaaaacttttataaaaagaccaacgggacccatcattcctaggataaccattttaagaatttttaaacctacaccaactatatcaaatgattttccaactctctcagttgggtttataagtttttttagagaatctatcacattttttattgctttaccgattttcatgatattttgctcacctttgacggtgaatgctccttcaagaaaattacctaattcgctcatatttttaattgaaccagacattatttttccgattgcctttatcccaagtttcatatcattaatttttccagtaaaattaatcattccttgccaaagactttcaggaaatagcttaataaattgagaccttaattccgcgattttctctcctggaccattggttagcatttcaacaaagcctttacttaaaagctttaatcctttaaatacttgatcaatgggtttcatgaacgattttaaagtcaacaccattccatcaaccttattcctaaaagtatctgaagttttataaaaatagatgaaagctgcgattaatgcaccaattccagcgataactacagcaaaaggatttgccatcatagccactttcattaatgagaaagccgttttgacaccattaattgctgttttaacactgttcataattccgatagttgctttgaaagcaataaagcctgctatagcaccaacaataacagaacctagaaatttaattatttgagcatttttactaataagttcaaacaatttatcaaaaataggaaatttatcgccaattgagtctaatagttctgcaaatgccgcttttacatgaggaatagaagctttgacaaaagtaacaatcgctcttggtaatgcttttaatatatttccaatcatgggaataaagttaccaaaaagaaatgtggccgttgtttctgctaactgatttaaagaaggttggatatcttgtccaagagccattttaccaagtacattagataaagaagctttcatagctgcgaatgaaccgctaagtgtctctgccgcttcttttgctgtagttcctgtgatatcaagattttcttgtattgcgtgaatggcattgtatacatcacttaggttactaatatcatatttaacgcctgtaagtttttcggcatcagagagtagtcgttgcatctcttcttttgtaccaccgtaacctaatttcaagttatcaagcattgtatagttttgctttgcaaaaccttgataggcattttgaatatcttgcatatttgtacccattttgttggcgttgtcagacatatcaatcatagccatatttgcaacatctgctgctttttctgtatcgccaccaacagattgtagtaaactagcgctaaaacttgttacgttttccatatatgcattcgctgataacccagatgttttatacgcttcatctgcatacttttttactttgtccgcactacctttgaataatgtttcaatcccaccgagggattgttgtagatttgctccttctgaaattgcggtagagaatgcagttttaattgatgaaatagctttgcctacaactgcagaagcaacttttactaatcccattgagatagctaaattttttatagaaaaaccagctttgtcactactgccttccaattgatctagctggctgttaagcccagctacacttttaccattaacatcaacatcaattacaacttgcccatcagccatcttcttcctcctctcctaagtcagtattaggtaaggcatatttcctttttaactctcgcattcgacgtttttctttacttgattcccctttttgaggttcccaattcctaatctgaataatcctctgcataatacaattatctggtagactctctaaaagtgcttgaaactcttcccaactcatttttccttgttcttcgaaaagattaataccaatttgacgaaaagaagcatagatatacttggcatcataaaccaaatctatatctttactatttattttagttggtaattcattgcctaattcatcagtttcaacttcggaatgttgacctaaaagcaggtattcatcataaacgtatttaaacatttcaaatttttgtagtatatctaaatcaacttccccaactaagattagaatcgaaagttcaatcttatcttctaatatcaaactctcatcttgtattatttcaaaaacatcaaggacatgatcaaacgctaagtttattggaaattctttgcccaaaaacgaaaaggatgtagtcagcggattattcaaccgcataatactcatcctttctttttgctaactttttttagatattcttgttttaatttttttgattttttaccacgctcatctaataattcagtctgtttcttagtgtattcttcaatagattcactaatacctactgataaatcgtaaaaggcgtttagccatgcgattatgtctgggatgtctttatacaaaattgcaaacgtaccttcaccaagtacggaatcatacccttttgtcaacacagaaatatataattcatcactcatttcctcagaattttcatctaagtctttcagttcttgtacgactttatcgtacgtttcttcatattcttttatatgctctaatgaacaatcaaagaaaaattcgtgcccagcaattttcactggaaaaccagtacgctctactttaatctctaatgctttcatttaattacctccataaataaggacagccaaatagctgcccttattttcgtatttatgcttgattattaagtgttaaggtatgttgtgctgttttcttaccatcctctgtttcgccttcggtaacataagtaccaactggtaccgtttctgtccaagtaatattacctgtttcagagacagcaaggccttctgttacaggcgtaa

SEQ ID NO: 52 - DNA, 1476 bp, Enterococcus faecalis
gttaaaatcctcacattcgataggaccaccgtcaatcgaataagcaaaaagtaaaatttcaaagttggggctatcggcgtatttataaaccccaactttggttaagtcttcatccgaataggtttcaatatcaatatttaacgttttgatgcaaaatacctactttcttgatataatttgtataaaatattttttaggaggggttaacttgattaaaaaaaatttctgtttcatacaatgaccacaaaagctctagagaacttgatattcctcagaaatgtgtgcactgtggcaaagatatgattccagacgttctaggtgcatattctaatgagaacgcctttgccacatacaccacaatcggtgtatttctcagatgtgtaaacgaagaatgtttgaaattttatgcattagagtatcaaggcggtcaatgtgttgagtattcatacaggccacctgtgaaaatagatttgcccgaaaatatagaaaaagtatctgaaactttcgtagatatatacacccaagccacaaccgctgaatctgaaatgctttcccaaatagcaggtgtaggataccgaaaatcattagaatttttaatcaaagattatgcgatacgtaaacaccctgatagagaaaactctataaagagaatgatgctaggtccagtaattggtgaatacttaagtgattttccaaaattacaaaatttagcaaaagcagctacttggataggtaacgatgagactcactatgttcgcaaacacacagataaagatattcaaaatatgaaggtctttattagagcggctgcacaatttattgcagctgactatgatgctgatatagctgaagaattcaccaatacttagagagccaatatattagactctcttttttaattcttcaataagaacacggtttgtttctaacgccttatctagcgaagctgaattaatttctaataaagatttcaaaaattcgacttcatttttcagctctgaaatctttattttttcgtaagtttcttcagtcacttccattttttcctacctcctgatcttctattggcgtatattcttgtttgacgtaaaacggaagatctgttaattcttcaaaatcaaatttttcacctataacgcttcttaagtcgtcatcatcttgaaaatagatagaatcaactttctccccttcaattagatcaaaagcatcttgtaattcgattggtaaataaattggacgtttacccatttaaatttctcctttcaaaagaaaaggggcgtttccgccccctattcagtttttagttaaacatgtcatcatcgttatcttcttcttcgtcatcccaatccaaatcaccgaagtcagattctgcatttgcacgaccgcccaagtattctcctttacatgttgttaaaatattatttaatccagcagtaatccctttgtttcctgaaaatccccgcaattaagccaaag

SEQ ID NO: 53 - DNA, 2127 bp, Enterococcus faecalis
aaaaaaataatgtttcatctcaatgatgcatatatcaataagttatatcctcgaaaaattactcaagaaaagaatctgctaaattcggcagattcttttcttacttataaagagtatttttttctgatttccattattttatttttctgttcaatgtttgttattgatgaaatttcatagtggtttaacaaactctctattccttcttttttaaaactatattttaatgctgaataagttactatttctacttctatgtttgtaagaaaacactcgatacctttaggtgttaggttaaataacattttttgataaaatggttttgcatttactgccacaccataagaattacctaaataacaagaaatgtttgtggatacatattcttctattattatttcaggaattggagatatttgtgttgatatctcatataaacgttctgcaaatggacgttcattataaaaattattaaaaccccaatgcgtttgttgcaaattataaatagcttttttaaaaattgctattttttcagtatcctttaaattacttaaaagacccaaattttcaaaaaataaacgagaatttgtagcactactcgcatctcccttaatcaaaatctcattatgtttttcaatcaatgttgttttcattttattgcttaaaataggttgaagtaattttgctaattctaatgagttagatcgcgtatgacttgcaatcgaagcatctataaatttagaatacaaaatttgaataatcaattctttctgagattcaaaagtatttaaaatcatttcggataacgttattagactctcttcactcgaatcttgattttctataacggttaaaaccttttttatgtctaccccaactatctcatgtacatcagataacccatacttacagcatctactaataaaagtaatcaactctctatcatcaatctctatttctgtaggatgtgctactgatgcattgtttctaatttctctacattagtttagataaaaatttccttcctcattaataatattgagctgataacatagttctattaattctgaatctgttattttatccaaaaaattatcatctttttttgccccagaaatattcttaatcatttccaatccaaaagaactaactttatttctcaattctttgatcacactattccatatgtatactattgcaccgtcaaacaatcctactgatatagccacacatgattttgctacgaacttatctataagtctagcaggaattaaattcaattctcggggtaacaaagataacgctatatttatatcttcattggatggaagtatatcccgagaaatagatagcgatgttgttatttgtgaaacaatatcctcactattttgagttagatttccagcatcaataacaagtgaaccatttgaaacagcaactaaattattcttattcatgaactaattaatctccttttttataatatttataagttataaactaatattattttaactaaatattaaaaaaaaaacaaatcaagctacaaattttttccatagatatagtaggagcaaccaatttaaaataaattattttaaaccgtacattcaaaaatcaataactgataacacatgtgctaacaatcattactttttgaacttacttactatgtaaaaaaaatattaaaaatcggcattctcaatacatttcaacagaaactactacccgaaaccataatctctgtatatttcacttataactacaactaaaacatgtagtaccttcgagttgccaaaagaacattattctaatttaagtagtgctatttaattatgtagtcttctctttcaaaatagggacgacttttagggacgacccttaaacattccataaaattttataaaatttattacaactatcaaagtatcaattacttatatatcaacactaccgacctccacaaaaaccatcaataaaattccgttcttgagacgggggttcgattccctcagggcgcgttatttaaatatgttgatttatcaacgtttctggacactctttattcatctaaagagtgtttttttcttgtttaggaatgatttttctatccgtattttcaatgtt

SEQ ID NO: 54 - DNA, 1314 bp, Enterococcus faecalis
tagcatcagaagatgtgaattgccgatgtacaacaattgaagttgtcgacggtatcagccctgaacttagaaaagataatgaatctaaagagatgtcggagtttaaaagctacgacgattggttagcttcaagattaagtaataatagcccagaagttattattcgtaataaattaaacgacattgatatgtcaaaagcaacaaaggatgatataatagagctaggaagtttaattaatcaacattaccaaatagataaaataattggcgacaagcaacggttgaaagacactttctcactatttagaaaaatgggtggagaaatcccttcagagagctgggcgaagggatcagagaaaaaagcgaaagaacaattaaataacgccttctcatattatcctaaaaattgggctgacttattggcaagaaatgataaaaaactattttcgggggttaataaaagaggctttttctttaacggtttgcttgataaaaggggacgatataaaacaaatattgaggggtttaaaacagattttgttaccatccatttatcgggtagggctacaaccccttttcatgagataggacatctgattgagtgttttaaccctgatgtcgtaagggtagaaaaagaatttatcgaagctcgaactagaggggagataccgactcgattgtcagaaatattcccagggagcggttatagcaatcgtgaagtaactaaaaaagataattttatcagtccatatataggtaaggtatatcctaatgccacagaagttttaagtgtcggtttggaaagtttatttgagccacagggcgggcagctaaaaagtataataaacggtaggccaataaaaaaaatatattacggatgatgaagaatttctcaatttagttatagggctaatattgaaagggtgagaaaatgagtaaagtacatgaagaatttattgaggctaaacaaatatacgaggctaagtttggcaaggacagtttggctcgtgttattttgcttgaccctttgggagcaaccgatgaggatttcactgaaagcacgagaaaattgaaaagttctgttaatcaaaataagcctttaaatcaaatagataaagatatatgggataaaattgtattttaagcatctagccaaaactggttaggtgctatttttgtacctaaaggaggccatgagatggaagatccttacgaccatttagacgcagactacgaagaatttttgagaaaggaaaaacctatggagaaaaaagtccaaactattaaagatatccaacgtaaaaaagagcgtgg

SEQ ID NO: 55 - DNA, 2243 bp, Enterococcus faecalis
gttttttataaaaaacaatgcttccatctttaattagcggttccattgaatctccctgaatattaatagcacaatctgctccttccggtacgtgtgcgaaagaatgctgttcaacgtctatatctccatattctagaactgcaggatttgctgcggtctttccgaccaatggaacaacttttctattgcgtgattccgtttcaaagttttgcttatctaattctttctcggcaaatattaatacatttttttgtcttttagtttctaacttagctgaggtttcatttattttcgttaatatttgagagaggggttcttcttcaaatcctaataggtattctggacttgtttctaaagctttggcaaaatcagatatcttattaacagggaattgtcttgttctattttcgtaacgtgacattgcggacttagctatccctactttttctgctaattcagtggatgtcatttttttctgtttgcgcaaatctgtaattaatgtcatgatctcatcgtttgttctcaaattattcacctcctaagagaataatactaataaagttcccaatagtcaacaaaaaatgttctaattcggcacaattagttgacaatcgggaattgtcgatatatcattaaagcacattcatggatgaataaaaatcaaggagggaattattaatatggaatttgaactcaatcgtttaaaggctgaaaggattgctaagggttattcgcaagaagaattagcaaaaaaattaggttggaccagaagtatgtatacaaaacgtgaaaatggtgcagtttcattgggagttgatgaactagcaaaaatagcaacagcattagaaatgccagaatctagaatatctatttttttttaatcgttgagttcccgaatgtcaacaaaggaggagggaagtgtgatatgaatcaaaagtcgtcagaaaaagtagatatcgatttattggcacaagcttttgctgatgttttaacgcgacgtactggcatacaacatgtttgtaaaataaaaagtaaaagtgaatctaagaaaagtgaaggtaacaattatcagaaagaagctaactaaaacttgtaaatatggggaatgcaacaaatacaaacaaaataactaaggaggcagaacatgaataacataaatgcgaaagagtaatttaattaaagaagaaaactaaacttggaggatcaaatgaataaaaaatttccatatcaaagaggagcgaaaaaacaacaaagaaagaaaaaacagacgttcaacagagtatctttttcagatggcaagatattattagatgattttcaacttttaggtgtaacagatttaaatttagaaagaagttcaactaatatagctgaacttcaattgagaatgatggtagacattaaggggttaacttgccacttataagattggtaattacgctggtagcaatatttgataatgcgtcaagagaaaagctacctacattagcggcaatttctttagtttttttccaattattttcggaacgaatatcattcaaaaattgatgaccttttggcgaaaggtcaacgcagtaaaagtctcctgacatgtcaaactctggattgtacaataattcgcttagagcggcttgtctgaaatgatagtacactgtatttttatcatattttttcaaatattcatcctgcggagaataggagtacattttgttagaatctgtactttgttcaatctccagcaataaatctcttacacagtcaggattcaatttcataatagtttcacctcgctttcaaaataattataccagaaaggaagtaaaccaaatgaacaatttagtaataatgaaagacaaataagcggtaacaagtagtttacaagttgctgaaacgtttagtaaaaatcaccaacatgttttgcgtgacttggataatttgaaagagggtgtccaaaattggacagacttattttacgaagacacttacattcatccacaaaacaaacaatcatatcgccaagtaattatgaaccgtgacggatttacatcactactagtcaatagcgttcacagtggcaaaaagctgattgcaattgctatcaatccaaagatattgaagcacgttaatcaaatggttaaaaagaaattcaacagccataaacttccaacctcaacaaagacaactaagcaatgctggctttatcagcaaatgaagaaacaaatgagcgtgtagatgtaattgaa

SEQ ID NO: 56 - DNA, 1502 bp, Enterococcus faecalis
attcaatggaaccgctaattaaagatggaagcattgttttttataaaaaacaatgatgatgttgaaaatggtgaaattgcaatcgtagaattgataatgatggtgttacatgtaagaaagtaattaaagactactcaaataaacaaattattttgagatcaatcaataccaaatatgaagatagaattttagaaaatgaaaaaattagaattattggaaaagttatattatagaacttttgtattagacttaaaagtattctattaaagaccaatctgtttaaaaaaataatttatagaagaatagttcgtggttagctaagaaataaatataaattttaaccctcggacttttcttcttacaaaaaaagaacatacattctctaaggagggatattatgaataaatatgaagtagaaaaaagactttgcgaagagttaaatattgaatatattaacttaagtttacgtacaggacctagtcatagatttactgaaaaagaataccaagaacttaaatctgattatgctcagttatttttacaattaaacaagcttaatgttgagaaagaccaagagtagtttttaaataagatagggggattcgtttatgctaaagagagcagcgttatacattagggtttctactgatcaacaagcaaaacacggggatagtttagacgcacaaatagctactttaaaagattatgtaagtactcaagacaatttgacaatcattgacacgtatattgatgacggcatttcaggacaaaaattgtaccgcgatgaatttcaacgcttattagaagatataaaaaagaacagaatagatattattttgtttaccaaattagatcgatggtttagaaatttacgtcattacttaaatattcaagaaatattagataattctggcgtaacatggcttgccgtttcacaaccatttttcaatactgatactgcttatgggcgttcctttgttaatcagtcaatgagttttgctgaattagaagcacaaatggcatctgaacgtatcaaagcagtatttgaaaacaaaattagaaagggagaagtggtaactggtagcgttccttttggttataaaatctgtgataagaagttaatacctaacgaaaatgcacctatagcaaaagacatttttaaacattattctattcacaacagtatacgcctaactgttgaatatctattcaatgaatatgatattacaagaagttctcgaacaatcaagcacatgttaaggaatagaaaatatataggtgaagtttctggtaacaaaaattattgtcctcccatagtagataaggaaacctttgagaaggttcaaaatctattagataaaaatatttcatctatagcaaaacgtacttatatcttttcaggactggtagtatgtagttgttgcggtaaaaaaatgactggacgttatcgaaaaagaaaatatattaaaaaagatggcacagtgatgtattatacaaaaaaa

SEQ ID NO: 57 - DNA, 1062 bp, Enterococcus faecalis
ctttagaaacagctagaaatattgcgaagaaatttaaggctagtttaaagcagaaaacggacctttatgtgattgagggaattttgattgatgctggttataaaaaagagccagtgaatttataagaagggagtggaggttttggtcgaccacaaagaatttttaatttgttttggggtaaggatacagtttttttaaatgaaaaaccagcaaaagctggtttaaggaatgttaagtttagatttcagggtagaagggactttgtcatcactgatgatttgccgttttataattcttttttcacttgcatttaaaagtagaatagttccttcatctaattttgatccatcagcataatccccagacgttacaacttcaaaatcaaaattacgactgttacctttagtgttaaatccaactactttatatttataacctttcccgaaaaatttaccactgtcatcttttaagtcagtttgttcaggtagaggagcctgtattttagcataatagtcttctcctacgtaagtagattgatagtaatcccaacctttccatccaataaatataagtaacgcaacaaaaattaagcttatgatttttttcatgtgaaaacctcctaaagtatttaatatatttaatttacatgaatcaaactaataagttaatcgaaataactaacatttttgtaataaatttattatccgattagaaagtgagtgaagaagatgattccaaaaatagaagtatggatgcatgatatgtccgttggctatcctgtgtggtttgaagtagattcaattgattatctagaaaattcgtttgttatagtagatgaatttggtagtccgcatgagttctctgctgaagggcgttcatttagagttaaggagggaacaaaatgagtagattaagcaaaataaatgatttggcatttgatctagtgcatgaatatatggaaccagccaaacaattttcttacggaaaacagatatatgatttatctgatgaaccagaactcaaatccaaatcagcagattgtgcttgattggttgaaagcaa

SEQ ID NO: 58 - DNA, 1037 bp, Enterococcus faecalis
tttttctctatttatcgctgttttataatgtttacattcaactagagttttatatgttacgcccattaatgaaaattcaatatacccatcaatctgatagtttccatcaaaggttttgataattttatcatgctctattatcaagttctcaagattttttgtctgttcagctaaaatatctagagagtagcgttcaaactgttctggtgttagttctaagtacttgtctatagttgggttaaacaaaatatacctcctttaaaaagactattaataaatctaaaatcttctatcttttgatagtctccagtaatgtaaacccaaagctgttagtttacacgatttgctttccattgcagcaaaatacatatgattagtatcaacaggttcgattaaaccaacactctcatataattgcagagttttaaatttagaaacattattttcatctgcatagggtttaattacttgatgtacttgagatggatcatttgtaaattcaaaagatggatctaagcgaaactcatcactaggatttgggaagaattgtgacaattgtcgcaaagttttctttgatactcttggttcaatagaacgtataggtaaaaaacttgaaatattagttttaaatactggacgttgttcccaagcacccaaagactgatctatgaatgagtataagctggctggtgttatttttccagttatatctgaggcgctacctcgcaagccttgtaataataaatttgtaaatacaccatgtcccgatgcttccatagatacttcattacgcatgcttgctgacattatcgttacaccttctccgagaaacgcttctgtaccttgaatgactccagattcacctactttaccagaaaaacaacaatctaaaataataattttgtttccgcaatttgaatggttagccaatctcaatatttctgtcattgggacacctaaatcacgacgtttaaagtcaggtgttaccaaataacctgatccttcatcacttccatgaccagaaaaataaaataatg

SEQ ID NO: 59 - DNA, 1719 bp, Enterococcus faecalis
caggactcgaacctgtgaccgaacggttatgagccgtttgctctaaccagctgagctaaaggtcctgaaagaatcctaatttactttaagactctttgtgtatttccgaaaaattttctggaagctctaatgatgatcctagcccttctttggttatgaacgtcagtactctatttaattctggaacaatatcttttaataaatctgttgaattatctatatctactaatgtcagattaggatcattattatcagtttcataaataccataaaaatttagttgcataaaagaatcttcacttttatctttatatagctcaacatgagcatctaattttaataattttgattcttctttgtttacctctactttaaattgtccatttactgatatactctcttcgatttcggccaagccaaactgaagagattcgattccactattgattcgtttgaatttgatttttcccatttcaattctcctttaatgcgattttcagtttcaaactccattacgttagcaattctaataatattatttcttagatctaactgacaaccgtatactttttgagctatttgttgatgttcagaatagctatttaatattagatcctccaataatttttctagtctattatatactttttctttaagaactttttttcccttaatttttacaatacatttgtaaaaggcatatgggtcatctgctagtcgtatcaaagaatcaatataattagcttgaatactaccgttctcaatagaagataagttactataactaatacccagcaatactgaaaaatctcttatagttagttcgtatttttctctaatttcttttacttttttagcagataacaaattgtgtctttctctgtaaatattatagtctttttctaaattttgatctgggttagaaaatggttcaaataattcatcatctgatattcttttataatattcatgtttgactttaaatgtatcgtttttaattgtaacgtcttcttctaaaaaaattacttcgtatctttcattttgattagtttcatatgaaaacttttctataattttagacatactaaatcacctctcccttatagtctaatggaaagcaatcaatttcttgttctctaggatgaaaagacatatatgctgcaacaaaattattctcttgtgcaatcaattccaacttcacatacacttttatttcatcccaaactaagccgaattctgtcacagttctattcgaggctctgtgatgctctgagggtcctctaaaataatgcttttcttctatattttctatgataaagttatgcattgcagcaacagttataccatatgcccttaagaatctagttgtttttgcattaacttcagaaaaacttacatgtccatcagaaacacattctttgaaagttgtgagaaaaaggttgactttagtaaataattcttcactcgacatttccacacctactattcacaaacttgttactcttgaataacataatatcacaatgggtaagtatctggcaagaatatttaaattcatgttacaaatctttaatttcataattacaaaaaagagccaccttggggaaggcgactaaaaaaacaaaatcacccacaatctgtcgtatgacggtgagtgattttgtcattaattgagattctatagtgaatgtcatcactaaagaaagtatactatacaga

SEQ ID NO: 60 - DNA, 1115 bp, Enterococcus faecalis
agattggctaggcagtctaatataaatctttagactactcaataaaaatgagtggtctttttttgtacgtaaaaaaaccactagattatgggtctagtggctaggtagcgttagtgtcaaaattaagatagattgtaatctctgaactcaagcttttaaaaaaggagtactaccatataaagagtataacaggtattaatgtatctgtcgcacatttacataaaattaattaggaggtggcgttgggtgtgcagaaatgggacttagcatatgagaattataaaaatggtatgaagtataaagatatagcagataaatacgatgtatctattaacaccgtgaagtcttggaaatctcgcaaatggaacgcgactcctaaagaggttgcaaccaaaaagaaaaaggttgcacacaaaaaagagtcgcaacccgttatagagaatgatgctttaacagagcaacagaaaatgttctgtttattttatttacagcattttaacgccactaaggcatatcaacaagcatacggatgtggctataattcagctagagccaatagcattaggttgctagcaaaagatcgcataaaaaaagagttgcaccgtttaaaagcagagttgcaacaagatgtgtttgtggatattaaagacttgatacaagagtatgttaagcaagcatttgccgatattaccgattttacagaatttggttatgatgaatatccgtttaaagatattaatggtgaagaggttatagacgaagaaacaggcgaagtaaaaacatacaaagtttcaaatgtctctcttaaagattcgaacgaggttgacggtacattgattcaggaagttaaaaaaggtaaagacggtgtatcagttaagctttacgataagcaaaaggctatgagtgagttgatgaagtatattgctactgatgaattaaaacaagcgcaaactgaaaaagctcaagcagaagcgaaaatacttacgaataaggctgataagttaaccgctggaggtaaagctaatgaattgctagaagccttgttagaagtaaaatcacgaggtgtgagcgatggcaattaattttagtgataagcaagtggcaaatatcaatta

SEQ ID NO: 61 - DNA, 20 bp, Artificial Sequence
  Forward Primer for Positive Control
aacgcttctttcctcccgag

SEQ ID NO: 62 - DNA, 20 bp, Artificial Sequence
  Reverse Primer for Positive Control
gtgtctcagtcccagtgtgg